(19) United States
(12) Patent Application Publication
(10) Pub. No.: US 2021/0190724 A1
(43) Pub. Date: Jun. 24, 2021

(54) METHOD, APPARATUS AND SYSTEM FOR SINGLE-MOLECULE POLYMERASE BIOSENSOR WITH TRANSITION METAL OR SILICON NANOBRIDGE

(71) Applicant: Roswell Biotechnologies, Inc., San Diego, CA (US)

(72) Inventors: Sungho Jin, San Diego, CA (US); Chulmin Choi, San Diego, CA (US); Paul Mola, San Diego, CA (US); Barry Merriman, San Diego, CA (US)

(73) Assignee: Roswell Biotechnologies, Inc., San Diego, CA (US)

(21) Appl. No.: 17/135,375

(22) Filed: Dec. 28, 2020

Related U.S. Application Data
(63) Continuation-in-part of application No. 16/840,755, filed on Apr. 6, 2020.
(60) Provisional application No. 62/954,292, filed on Dec. 27, 2019.
(60) Provisional application No. 62/830,231, filed on Apr. 5, 2019.

(51) Int. Cl. G01N 27/414; C12Q 1/00

(52) U.S. Cl. CPC G01N 27/4145; C12Q 1/001; G01N 27/4146

(57) ABSTRACT

Various aspects of the present disclosure provide methods, apparatus and systems for single-molecule biosensors having transition metal dichalcogenide (TMD), Si or Si-semiconductor nanobridge for sequencing, information storage and reading. In various embodiments, the present disclosure provides nanofabrication of biomolecular sensing devices beginning with a silicon-on-insulator (SOI) wafer and in general to the fabrication of biosensor devices for analyzing DNA and related biomolecules.

### CROSS-REFERENCE TO RELATED APPLICATIONS

This application is a continuation-in-part of U.S. patent application Ser. No. 16/840,755 filed Apr. 6, 2020, entitled “Method, Apparatus and System for Single-Molecule Polymerase Biosensor with Transition Metal Nanobridge.” This application also claims priority to, and the benefit of, U.S. Provisional Patent Application Ser. No. 62/954,292 filed Dec. 27, 2019, entitled “Pre-Aligned and Transferred, SOI Based Nano-Bridge Biosensor Array and Memory, Comprising Single Biomolecule Sensor, and Method and Uses Thereof.” The '755 Application claims priority to, and the benefit of, U.S. Provisional Patent Application Ser. No. 62/830,231 filed Apr. 5, 2019, entitled “Single-Molecule Polymerase Biosensor Comprising Transition Metal Dichalcogenide Nanobridge for Sequencing, Information Storage and Reading.” These disclosures are incorporated herein by reference in their entireties for all purposes.

### FIELD

The disclosure relates to biosensors, and in particular to methods, apparatus and systems for single-molecule biosensors having a transition metal dichalcogenide or silicon nanobridge for sequencing, information storage and reading.

### BACKGROUND

Analysis of biomolecules including DNAs and genomes has received an increasing amount of attention in recent years in various fields of precision medicine or nanotechnology. The seminal work of Maclyn McCarty and Oswald T. Avery in 1946 (see, “Studies On The Chemical Nature Of The Substance Inducing Transformation Of Pneumococcal Types II. Effect Of Desoxyribonuclease On The Biological Activity Of The Transforming Substance,” The Journal of Experimental Medicine 83(2), 89-96 (1946)), demonstrated that DNA was the material that determined traits of an organism. The molecular structure of DNA was then first described by James D. Watson and Francis HC Crick in 1953 (see a published article, “Molecular structure of nucleic acids.”, Nature 171,737-738 (1953)), for which they received the 1962 Nobel Prize in Medicine. This work made it clear that the sequence of chemical letters (bases) of the DNA molecules encode the fundamental biological information. Since this discovery, there has been a concerted effort to develop means to actually experimentally measure this sequence. The first method for systematically sequencing DNA was introduced by Sanger, et al in 1978, for which he received the 1980 Nobel Prize in Chemistry. See an article, Sanger, Frederick, et al., “The nucleotide sequence of bacteriophage φX174.” Journal of Molecular Biology 125, 225-246 (1978).

Sequencing techniques for genome analysis evolved into utilizing automated commercial instrument platform in the late 1980's, which ultimately enabled the sequencing of the first human genome in 2001. This was the result of a massive public and private effort taking over a decade, at a cost of billions of dollars, and relying on the output of thousands of dedicated DNA sequencing instruments. The success of this effort motivated the development of a number of “massively parallel” sequencing platforms with the goal of dramatically reducing the cost and time required to sequence a human genome. Such massively parallel sequencing platforms generally rely on processing millions to billions of sequencing reactions at the same time in highly miniaturized microfluidic formats. The first of these was invented and commercialized by Jonathan M. Rothberg's group in 2005 as the 454 platform, which achieved thousand fold reductions in cost and instrument time. See, an article by Marcel Margulies, et al., “Genome Sequencing in Open Microfabricated High Density Picoliter Reactors,” Nature 437, 376-380 (2005). However, the 454 platform still required approximately a million dollars and took over a month to sequence a genome.

The 454 platform was followed by a variety of other related techniques and commercial platforms. See, articles by M. L. Metzker, “Sequencing Technologies—the Next Generation,” Nature Reviews Genetics 11(1), 31-46 (2010), and by C. W. Fuller et. al, “The Challenges of Sequencing by Synthesis,” Nature Biotechnology 27(11), 1013-1023 (2009). This progress lead to the realization of the long-sought “$1,000 genome” in 2014, in which the cost of sequencing a human genome at a service lab was reduced to approximately $1,000, and could be performed in several days. However, the highly sophisticated instrument for this sequencing cost nearly one million dollars, and the data was in the form of billions of short reads of approximately 100 bases in length. The billions of short reads often further contained errors so the data required interpretation relative to a standard reference genome with each base being sequenced multiple times to assess a new individual genome.

Thus, further improvements in quality and accuracy of sequencing, as well as reductions in cost and time are still needed. This is especially true to make genome sequencing practical for widespread use in precision medicine (see the aforementioned article by Fuller et al), where it is desirable to sequence the genomes of millions of individuals with a clinical grade of quality.

While many DNA sequencing techniques utilize optical means with fluorescence reporters, such methods can be cumbersome, slow in detection speed, and difficult to mass produce to further reduce costs. Label-free DNA or genome sequencing approaches provide advantages of not having to use fluorescent type labeling processes and associated optical systems, especially when combined with electronic signal detection that can be achieved rapidly and in an inexpensive way.

In this regard, certain types of molecular electronic devices can detect single molecule, biomolecular analytes such as DNAs, RNAs, proteins, and nucleotides by measuring electronic signal changes when the analyte molecule is attached to a circuit, for example, a field effect transistor device. Such methods are label-free and thus avoid using complicated, bulky and expensive fluorescent type labeling apparatus. These methods can be useful for lower cost sequencing analysis of DNA, RNA and genome.

Certain types of molecular electronic devices can detect the biomolecular analytes such as DNAs, RNAs, proteins, and nucleotides by measuring electronic signal changes when the analyte molecule is attached to the circuit comprising a pair of conductive electrodes. Such methods are label-free and thus avoids using complicated, bulky and expensive fluorescent type labeling apparatus. One example of sequencing-by-synthesis approach is to utilize a single molecule polymerase with incorporated DNAs, the sequence of which is detected through a current pulse signal when each type of the nucleotides (A,T,C, G) is attached to the polymerase complex with a distinct electrical signal.

While current molecular electronic devices can electronically measure molecules for various applications, they lack the reproducibility as well as scalability and manufacturability needed for rapidly sensing many analytes at a scale of up to millions in a practical manner. Such highly scalable methods are particularly important for DNA sequencing applications, which often need to analyze millions to billions of independent DNA molecules. In addition, the manufacture of current molecular electronic devices is generally costly due to the high level of precision needed.

### SUMMARY

Disclosed herein are principles that provide new and improved sequencing apparatuses, device structures and methods using two-dimensional layer structured semiconductors, which can provide reliable DNA genome analysis performance and are amenable to scalable manufacturing. In various embodiments, the present disclosure provides nanofabrication of biomolecular sensing devices and fabrication of devices for analyzing DNA and related biomolecules. In various embodiments, the present disclosure provides DNA-based memory systems.

In various embodiments herein, biomolecular sensors comprise a nanobridge structure disposed over a nanogap, wherein the nanobridge comprises a transition metal dichalcogenide (TMD) material or a silicon material, e.g., pure crystalline silicon or various doped silicon semiconductor materials.

Two dimensional (2D) layered transition metal dichalcogenides (TMDs) materials and devices have attracted a great deal of interest due to their novel electronic, physical and chemical characteristics. One example is MoS₂ which can be incorporated as a sensor device. MoS₂ type 2D materials can be a single layered material or several layered material, and can be obtained by various techniques, such as e.g., by isolation of very thin MoS₂ layer through mechanical exfoliation, physical or chemical vapor deposition, molecular beam epitaxy (MBE) type construction or sulfurization of transition metal layer such as Mo or W.

Transition metal dichalcogenide (TMD) monolayers are in general atomically thin semiconductors of the type MX₂, which M a transition metal atom (notably Mo, W, or Ti, Zr, Hf, V, Nb, Ta, Tc, Re, Co, Rh, Ir, Ni, Pd, Pt) and X a chalcogen atom (S, Se, or Te). One layer of M atoms is sandwiched between two layers of X atoms. A MoS₂ monolayer can be about 6.5 Å thick. TMD monolayers of e.g., MoS₂, WS₂, MoSe₂, WSe₂, MoTe₂ have a direct band gap, and can be used in electronics as transistors or sensors. Either monolayer TMD or few layer TMD can be structurally modified, to be utilized as solid state DNA or genome sensors. Being an ultrathin direct bandgap semiconductor, a transition metal dichalcogenide such as single-layer MoS₂ may have potential for widespread applications in nanoelectronics, optoelectronics, and energy harvesting.

The layered TMDs typically have a hexagonal type structure with space group P63/mmc. It should be noted that monolayers of TMD materials are not just one atom thick as graphene, but are made up with tri-atomic thick layers consisting of metal atoms (such as Mo or W) sandwiched between two layers of chalcogen atoms (such as S, Se, or Te). The atoms in-plane in MoS₂ type 2D materials are put together and bonded by strong covalent bonds. The adjacent layers of TMD like MoS₂ along the thickness direction are joined together by a weak van der Wall force binding. This force is strong enough to hold the layers together with mechanical integrity. The TMD materials provide interesting and unique possibilities to design electronic devices involving hetero structures. The direct band gap of TMD monolayers is tunable with the application of the mechanical strain.

Single nucleotide identification and DNA sequencing have already been demonstrated with biological nanopores or solid state nanopores such as those in graphene and MoS₂ layers. A DNA type molecule is threaded through a nanopore under an applied electric field, so that the sequence of nucleotides is read by monitoring small changes in the ionic current flowing through the pore, which are induced by individual nucleotides temporarily residing within the pore during threading. However, the fragility of such pores, together with difficulties related to reproducible and low noise measurement of detection signals in nanopore sequencing methods in general are some of the current issues that need to be addressed.

The disclosed principles provide, among others, new biomolecular sensor devices and associated methods, employing transition metal dichalcogenide nanoribbons as a component of molecular bridge, which in turn comprises an attached, preferably single molecule polymerase to analyze DNA lines or fragments by step by step attachments of nucleotides or short DNA fragments.

Various embodiments are disclosed herein regarding specially processed, 2D layer-containing enzyme polymerase sensor device structures and methods of manufacture for a multitude of devices for use in electronic DNA, RNA or genome sequencing systems. Unique geometrical modifications are made so as to enable a construction of sensor device comprising only a single molecule polymerase enzyme for more accurate electronic analysis. Such label-free, single molecule based sequencing analysis systems utilize preferably a nanoscale dimension-controlled, transition metal dichalcogenide (TMD) micro-ribbon or nano-ribbon bridge. The electronic system may also be used in analyzing other types of biomolecules, such as proteins, depending on how the molecular sensors are functionalized to interact with biomolecule sensing targets. The TMD-based sequencing systems disclosed here can be assembled into a massively parallel configuration for rapid analysis of targets including nucleotides, in particular for applications to sequencing of a DNA molecule, or a collection of such molecules constituting an entire human genome. Such systems in the present disclosure can also be used for DNA-based information storage, for example, for archival storage of huge volume of information in human society.

In various embodiments, a single-molecule biosensor comprises: a conducting electrode pair disposed on a substrate, the electrode pair comprising a first electrode and a second electrode spaced-apart from the first electrode by a nanogap of fixed width; a TMD, Si, or doped Si-semiconductor layer disposed on the substrate electrically connecting the first and second electrodes; a dielectric layer disposed completely over the first and second electrodes to encase the first and second electrodes, and disposed partially over the TMD, Si, or doped Si-semiconductor layer so as to leave an exposed portion of the TMD, Si, or doped Si-semiconductor layer having a width less than the width of the nanogap; an enzyme molecule attached to the exposed portion of the TMD, Si, or doped Si-semiconductor layer; and a microfluidic system encasing the conducting electrode pair, the contiguous TMD layer and the enzyme molecule attached thereto.

In various embodiments, the TMD layer comprises at least one TMD having a structure MS₍₂₋ₓ₎, MS₍₂₊ₓ₎, MSe₍₂₋ₓ₎, MSe₍₂₊ₓ₎, MTe₍₂₋ₓ₎ or MTe₍₂₊ₓ₎, wherein x is 0-0.3, and M is Mo, W, Ti, Zr, Hf, V, Nb, Ta, Tc, Re, Co, Rh, Ir, Ni, Pd, or Pt.

In various embodiments, the TMD layer comprises a TMD selected from the group consisting of MoS₂, WS₂, TiS₂, ZrS₂, HfS₂, VS₂, NbS₂, TaS₂, TcS₂, ReS₂, CoS₂, RhS₂, IrS₂, NiS₂, PdS₂, PtS₂, MoSe₂, WSe₂, TiSe₂, ZrSe₂, HfSe₂, VSe₂, NbSe₂, TaSe₂, TcSe₂, ReSe₂, CoSe₂, RhSe₂, IrSe₂, NiSe₂, PdSe₂, PtSe₂, MoTe₂, WTe₂, TiTe₂, ZrTe₂, HfTe₂, VTe₂, NbTe₂, TaTe₂, TcTe₂, ReTe₂, CoTe₂, RhTe₂, IrTe₂, NiTe₂, PdTe₂, PtTe₂, and mixtures thereof.

In various embodiments, the TMD layer comprises a mixed TMD compound selected from the group consisting of Mo(SₓSe_{y}Te_{z})₂, W(SₓSe_{y}Te_{z})₂, Ti(SₓSe_{y}Te_{z})₂, Zr(SₓSe_{y}Te_{z})₂, Hf(SₓSe_{y}Te_{z})₂, V(SₓSe_{y}Te_{z})₂, Nb(SₓSe_{y}Te_{z})₂, Ta(SₓSe_{y}Te_{z})₂, Tc(SₓSe_{y}Te_{z})₂, Re(SₓSe_{y}Te_{z})₂, Co(SₓSe_{y}Te_{z})₂, Rh(SₓSe_{y}Te_{z})₂, Ir(SₓSe_{y}Te_{z})₂, Ni(SₓSe_{y}Te_{z})₂, Pd(SₓSe_{y}Te_{z})₂, Pt(SₓSe_{y}Te_{z})₂, and mixtures thereof, wherein (x+y+z) is 0.7-1.3.

In various embodiments, the TMD layer comprises at least one TMD compound of structure M_{(1-w)}N_{y}X_{(2-z)}Y_{z}, wherein M is Al, Si, Ga, Ge, In, Sn, Sb, Bi, Na, K, Ca, Mg, Sr, or Ba; X is S, Se, or Te; Y is Li, B, C, N, O, P, F, Cl, or I; w is 0-0.3; and z is 0-0.3.

In various embodiments, the substrate comprises Si, SiO₂ on Si, or Al₂O₃ on Si.

In various embodiments, the pair of conducting electrodes comprise at least one of Au, Ag, Pd, Pt, Ru, Rh, Al, Cu, Ni, or alloys thereof.

In various embodiments, the Si, or doped Si-semiconductor layer comprises single crystalline silicon, n-type doped silicon, or p-type doped silicon.

In various embodiments, the width of the nanogap is from about 20 nm to about 100 nm, and wherein the width of the exposed portion of the TMD, Si, or doped Si-semiconductor layer is about 5 nm.

In various embodiments, the enzyme molecule comprises a DNA polymerase or an RNA polymerase.

In various embodiments, a DNA or RNA sequencing device comprises: a pair of electrodes disposed on a substrate, the pair comprising a first electrode and a second electrode spaced-apart from the first electrode by a nanogap having a width W1; a first nanopillar electrically attached to the first electrode and a second nanopillar electrically attached to the second electrode, wherein the two nanopillars are separated by a width W2 and wherein W2≥W1; a dielectric layer disposed over the electrode pair and over the nanogap to surround the nanopillars such that only a top surface of each nanopillar is exposed; a TMD, Si, or doped Si-semiconductor layer disposed on the dielectric layer and over the exposed top surface of each nanopillar, electrically connecting the first and second nanopillars; an enzyme molecule attached to a region of the TMD, Si, or doped Si-semiconductor layer between the nanopillars and directly over the nanogap; and a microfluidic system encasing the electrode pair, the TMD, Si, or doped Si-semiconductor layer and the enzyme molecule attached thereto.

In various embodiments, the TMD layer comprises at least one TMD having a structure MS₍₂₋ₓ₎, MS₍₂₊ₓ₎, MSe₍₂₋ₓ₎, MSe₍₂₊ₓ₎, MTe₍₂₋ₓ₎ or MTe₍₂₊ₓ₎, wherein x is 0-0.3, and M is Mo, W, Ti, Zr, Hf, V, Nb, Ta, Tc, Re, Co, Rh, Ir, Ni, Pd, or Pt.

In various embodiments, the TMD layer comprises a TMD selected from the group consisting of MoS₂, WS₂, TiS₂, ZrS₂, HfS₂, VS₂, NbS₂, TaS₂, TcS₂, ReS₂, CoS₂, RhS₂, IrS₂, NiS₂, PdS₂, PtS₂, MoSe₂, WSe₂, TiSe₂, ZrSe₂, HfSe₂, VSe₂, NbSe₂, TaSe₂, TcSe₂, ReSe₂, CoSe₂, RhSe₂, IrSe₂, NiSe₂, PdSe₂, PtSe₂, MoTe₂, WTe₂, TiTe₂, ZrTe₂, HfTe₂, VTe₂, NbTe₂, TaTe₂, TcTe₂, ReTe₂, CoTe₂, RhTe₂, IrTe₂, NiTe₂, PdTe₂, PtTe₂, and mixtures thereof.

In various embodiments, the TMD layer comprises a mixed TMD compound selected from the group consisting of Mo(SₓSe_{y}Te_{z})₂, W(SₓSe_{y}Te_{z})₂, Ti(SₓSe_{y}Te_{z})₂, Zr(SₓSe_{y}Te_{z})₂, Hf(SₓSe_{y}Te_{z})₂, V(SₓSe_{y}Te_{z})₂, Nb(SₓSe_{y}Te_{z})₂, Ta(SₓSe_{y}Te_{z})₂, Te(SₓSe_{y}Te_{z})₂, Re(SₓSe_{y}Te_{z})₂, Co(SₓSe_{y}Te_{z})₂, Rh(SₓSe_{y}Te_{z})₂, Ir(SₓSe_{y}Te_{z})₂, Ni(SₓSe_{y}Te_{z})₂, Pd(SₓSe_{y}Te_{z})₂, Pt(SₓSe_{y}Te_{z})₂, and mixtures thereof, wherein (x+y+z) is 0.7-1.3.

In various embodiments, the TMD layer comprises at least one TMD compound of structure M_{(1-w)}N_{y}X_{(2-z)}Y_{z}, wherein M is Al, Si, Ga, Ge, In, Sn, Sb, Bi, Na, K, Ca, Mg, Sr, or Ba; X is S, Se, or Te; Y is Li, B, C, N, O, P, F, Cl, or I; w is 0-0.3; and z is 0-0.3.

In various embodiments, the dielectric layer comprises PMMA, SiO₂ or HSQ.

In various embodiments, the first and second electrodes comprise at least one of Au, Ag, Pd, Pt, Ru, Rh, Al, Cu, Ni, or alloys thereof, and wherein the first and second nanopillars comprise Au, Pd, Pt, Ru, or Rh.

In various embodiments, the Si, or doped Si-semiconductor layer comprises single crystalline silicon, n-type doped silicon, or p-type doped silicon.

In various embodiments, W1 is from about 5 nm to about 20 nm, and W2 is from about 5 nm to about 100 nm.

In various embodiments, the enzyme molecule comprises a DNA polymerase or an RNA polymerase.

### BRIEF DESCRIPTION OF THE DRAWING FIGURES

The features and advantages of embodiments of the present disclosure will become more apparent from the detailed description set forth below when taken in conjunction with the drawings. The drawings and the associated descriptions are provided to illustrate embodiments of the disclosure and not to limit the scope of what is claimed.

FIGS. 1A and 1B (Design option #1) depict a side view for fabrication steps for MoS₂ ribbon bridge based biosensor, with size-limited, MoS₂ or TMD or semiconductor islands exposed, with optionally defective MoS₂, and any damage by nanopatterning repaired by annealing or plasma-treatment. (a) deposit and pattern conducting electrode pair (source and drain) and place a suspended MoS₂ layer, (b) To enable Nanoimprinting, planarize the surface, then use vertical Au nanopillar, and use size-limited MoS₂ region, (c) allow preferably a single molecule enzyme (DNA, RNA polymerase, etc.) to be attach on exposed MoS₂, (using biotin-streptavidin, antigen-antibody, peptide complex, etc.) for polymerase reaction of nucleotide attachment for FET sequencing, or other protein sensing.

FIGS. 2A and 2B (Design option #2) depict an exemplary design of single molecule bridge of DNA or RNA sensor comprising size-limited, MoS₂ type 2D chalcogenide semiconductor nano-ribbon region. The polymerase reaction of nucleotide attachment (nucleotide monomers like A,T,C,G, etc.) alters the electrical current properties of the FET molecular bridge on pulsing for sequencing analysis.

FIGS. 3A and 3B (Design option #3) depict an alternative design using temporary, removable guiding channel to allow a single polymerase attachment on MoS₂ nanoribbon bridge, but can be dissolved away later. (a) Removable tapered channel prepared by microfabrication or nanoimprinting, (b) Guided single polymerase attachment onto MoS₂ nanoribbon bridge, followed by dissolution removal of the sacrificial guide structure above.

FIGS. 4A and 4B schematically illustrate a cross-sectional view of producing diameter-reduced-tip Au electrode for size-confined molecular bridge sensor formation.

FIGS. 5A and 5B depict schematically a sectional view showing an example process of using a sacrificial plug to create a 5-10 nm, size-limited structure to place a single molecule polymerase sensor. Either nanoimprinting or sequential multilayer resists having gradually different dissolution rate to create a funnel shape type guidance geometry.

FIG. 6 provides an alternative method of guidance funnel shaping using nanoimprinting for 5-10 nm regime, in the PMMA or HSQ type resist to enable a placement of a single molecule polymerase sensor on a MoS₂ nanoribbon bridge. The protruding PMMA structure above also protects the attached polymerase from mechanically washed away by microfluidic solution flow or post-sequencing washing operation.

FIG. 7 illustrates use of additional force parameters to assist in guidance and placement of single polymerase or single biosensor molecule per each of the electrode array structure.

FIGS. 8A and 8B (Design option #4) depict an exemplary design of utilizing size-confined DNA assembly well to have only a “Single Streptavidin” immobilized for sequencing or protein sensing.

FIG. 9 (Design option #5) depicts a cross-sectional view of example design of utilizing shape memory type metal, ceramic or polymer functional material for temperature-, magnetic-, E-field-, pH- or chemical-responsive, dimension-changeable structure to physically trap only a “Single Streptavidin” or “single Polymerase” for high conductivity sequencing or protein sensing.

FIG. 10 depicts a top view of an array of TMD bridges like MoS₂ or WS₂, bridge molecular sensors, with a size-limiting structure for single polymerase. Massively parallel electronic sequencing analysis can be performed with many devices organized into a system, having as many as 10,000 or even at least 1 million devices.

FIG. 11 depicts MoS₂ ribbon transfer deposited. A large sheet MoS₂ (e.g., >1 cm²) can be transfer deposited on CMOS chip or onto another device surface, and e-beam or nano-imprint patterned. For less time-consuming process/assembly, pre-slitted (but still frame-attached) MoS₂ could be transferred, or an array of nano-ribbons can be wet-stamp transferred onto the device surface, preferably using an elastomeric stamp (e.g., PDMS based), with optional binding-assisting thin layer of polymer type material, which can be later washed or burned away.

FIGS. 12A and 12B depict MoS₂ nano-ribbon array patterned on flat SiO₂ or other removable substrate surface, to be made transferable and releasable by a sequence of processes.

FIG. 13 depicts transfer of MoS₂ nanoribbons by PDMS type soft stamp. The PDMS stamp optionally has protruding ridges for easier pick up of the nanoribbons.

FIG. 14 depicts a top view of an array of Au electrode pairs (or other metal) with float-transferred or PDMS stamp-transferred MoS₂ bridges, with redundant ribbons to ensure at least one bridge formation occurs. Optionally, a masking coating with blocking agent may be added to prevent biotin, streptavidin or polymerase adhesion, except for a local ˜5 nm circle on MoS₂ surface, so as to ensure only a single enzyme molecule is attached.

FIG. 15 depicts: (a) Tethered array of encoded (memory written) DNA fragments periodically positioned on a substrate, (b) Polymerase-MoS₂ nanobridge array approaching DNA array being released, (c) DNA templates are taken up by polymerase array and the completed sensor array is moved array for DNA sequencing to read the recorded memory information. (Microfluidics chamber not shown). Massive parallel DNA written information array in combination with massively parallel MoS₂ nanobridge reader array allows ultrafast, “random-access-enabled” DNA memory retrieval. Several different configuration of tethered DNA memory array, with various methods of tethering and releasing, can be made to maximize the DNA memory processing capability.

FIG. 16 illustrates a sensor array fabrication method in accordance with various embodiments of the present disclosure, beginning with a silicon-on-insulator (SOI) wafer.

FIG. 17 illustrates various dimensions and shapes for semiconductor nanobridges in accordance with various embodiments, which are controllable, for example, by nanofabrication, nanoimprinting, shadow-mask RIE etch or by repeated oxidation/chemical etch.

FIG. 18 illustrates a method of alignment and attachment of floating nanoribbons comprising applying an electrical field across the electrode array to attract and attach one nanoribbon to each electrode pair.

### DETAILED DESCRIPTION

Various aspects of the present disclosure generally provide sequencing apparatus, device structures, and methods for using two-dimensional, layer structured semiconductors usable to provide DNA and genome analysis performance. Various disclosed embodiments are amenable to scale-up processes in commercial manufacturing.

In various embodiments of the present disclosure, a biomolecular sensor comprises a nanobridge connected to spaced-apart electrodes and suspended over a gap between them. In various embodiments, the nanobridge comprises a transition metal dichalcogenide material or a silicon material. In various embodiments, a silicon material herein may include pure crystalline silicon or any type of doped silicon semiconductor material. Such materials may be obtained from silicon-on-insulator wafers.

Two dimensional (2D) layered materials such as transition metal dichalcogenides (TMDs) materials and devices have received much attention in recent years by virtue of their unique electronic, physical and chemical properties. One example is molybdenum dichalcogenide MoS₂ which can be incorporated as a sensor device. MoS₂ type 2D materials can be a single layered material or several layered material. The 2D layer materials such as MoS₂ can be produced by various known techniques, e.g., by isolation of very thin MoS₂ layer through mechanical exfoliation, physical or chemical vapor deposition, molecular beam epitaxy (MBE) type construction, or sulfurization of a transition metal layer such as Mo or W.

### Definitions

As used herein, the term “nucleotide” means either the native dNTPs like A, T, C, G (i.e., dATP, dTTP, dCTP and dGTP), or collectively refers to various types of modified dNTPs.

As used herein, the term “polymerase” means an enzyme that synthesizes long chains or polymers of nucleic acids. For example, DNA polymerase and RNA polymerase can copy a DNA or RNA template strand, respectively, using base-pairing interactions, thus assembling DNA and RNA molecules.

### TMD Layers and Combined TMD Materials for Sensor Bridges

In various embodiments, a TMD layer is incorporated as a part of sensor bridge structure to attach an enzyme type biomolecule to attract various types of nucleotides for electronic detection signals.

Two dimensional transition metal dichalcogenide (TMD) monolayers are in general atomically thin semiconductors of the type MX₂, with M a transition metal atom (notably including Mo, W, or Ti, Zr, Hf, V, Nb, Ta, Tc, Re, Co, Rh, Ir, Ni, Pd, Pt) and X a chalcogen atom (such as S, Se, or Te.). One layer of M atoms is sandwiched between two layers of X atoms. Both the transition metal and the chalcogenide element can be partly replaced (or doped) with other elements. Therefore, the two dimensional TMD layer incorporated into the molecular sensor bridge construction can have various modified or altered composition ranges, including the following:

(i) MoS₂, WS₂, or TiS₂, ZrS₂, HfS₂, VS₂, NbS₂, TaS₂, TcS₂, ReS₂, CoS₂, RhS₂, IrS₂, NiS₂, PdS₂, PtS₂ and their modifications or combinations, including modified stoichiometry of sulfur contents having MX₍₂₋ₓ₎ or MX₍₂₊ₓ₎ with the desired value of x in the range of 0-1.0, preferably in the range of 0-0.5, even more preferably 0-0.3. For various embodiments, the sulfur stoichiometry is intentionally altered so as to provide vacancy defects, interstitial defects, and aggregated defects so as to increase the surface energy and enhance the adhesion of biomolecule to the bridge sensor. Such defects can also increase the bandgap for stronger sensor signals;

(ii) MoSe₂, WSe₂, or TiSe₂, ZrSe₂, HfSe₂, VSe₂, NbSe₂, TaSe₂, TcSe₂, ReSe₂, CoSe₂, RhSe₂, IrSe₂, NiSe₂, PdSe₂, PtSe₂ and their modifications or combinations, including modified stoichiometry of selenium contents having MX₍₂₋ₓ₎ or MX₍₂₊ₓ₎ with the desired value of x in the range of 0-1.0, preferably in the range of 0-0.5, even more preferably 0-0.3. For various embodiments, the selenium stoichiometry is intentionally altered so as to provide vacancy defects, interstitial defects, and aggregated defects so as to increase the surface energy and enhance the adhesion of biomolecule to the bridge sensor. Such defects can also increase the bandgap for stronger sensor signals;

(iii) MoTe₂, WTe₂, or TiTe₂, ZrTe₂, HfTe₂, VTe₂, NbTe₂, TaTe₂, TcTe₂, ReTe₂, CoTe₂, RhTe₂, IrTe₂, NiTe₂, PdTe₂, PtTe₂ and their modifications or combinations, including modified stoichiometry of tellurium contents having MX₍₂₋ₓ₎ or MX₍₂₊ₓ₎ with the desired value of x in the range of 0-1.0, preferably in the range of 0-0.5, even more preferably 0-0.3. For various embodiments, the tellurium stoichiometry is intentionally altered so as to provide vacancy defects, interstitial defects, and aggregated defects so as to increase the surface energy and enhance the adhesion of biomolecule to the bridge sensor. Such defects can also increase the bandgap for stronger sensor signals;

(iv) Mixed TMD compounds in which the MX₂ compound has mixed metals and/or mixed chalcogenide. For example Mo(SₓSe_{y}Te_{z})₂, W(SₓSe_{y}Te_{z})₂, or Ti(SₓSe_{y}Te_{z})₂, Zr(SₓSe_{y}Te_{z})₂, Hf(SₓSe_{y}Te_{z})₂, V(SₓSe_{y}Te_{z})₂, Nb(SₓSe_{y}Te_{z})₂, Ta(SₓSe_{y}Te_{z})₂, Tc(SₓSe_{y}Te_{z})₂, Re(SₓSe_{y}Te_{z})₂, Co(SₓSe_{y}Te_{z})₂, Rh(SₓSe_{y}Te_{z})₂, Ir(SₓSe_{y}Te_{z})₂, Ni(SₓSe_{y}Te_{z})₂, Pd(SₓSe_{y}Te_{z})₂, Pt(SₓSe_{y}Te_{z})₂ where the combined (x+y+z) is 1-3, preferably 0.5-1.5, even more preferably 0.7-1.3. Alternatively, two or more metals can be combined for sulfur containing, Se-containing or Te-containing TMD layers, e.g., (MoₓW_{y}Co_{z})S₂, (HfₓW_{y}Co_{z})Te₂ and so forth; or

(v) M_{(1-w)}N_{y}X_{(2-z)}Y_{z} structure in which the transition metal M is partially substituted with non-transition elements N, with a concentration of w and the N element selected from one or more of Al, Si, Ga, Ge, In, Sn, Sb, Bi, Al, Na, K, Ca, Mg, Sr, Ba, with the w value in the range of 0-0.3, and the chalcogenide element X partially substituted with a non-chalcogenide element Y, with the Y element selected from one or more of Li, B, C, N, O, P, F, Cl, I, with the z value in the range of 0-0.3.

In various embodiments, the thickness of a MoS₂ monolayer can be about 6.5 Å. The TMD materials in their simplest monolayer structure, e.g., MoS₂, WS₂, MoSe₂, WSe₂, MoTe₂, have a direct band gap, and hence can be used in electronics as transistors or sensors. Either monolayer TMD or few layer TMD can be structurally modified, to be utilized as solid state DNA or genome sensors, without labeling with optical capability. Being an ultrathin direct bandgap semiconductor, a transition metal dichalcogenide such as single-layer MoS₂ has found some useful applications in nanoelectronics, optoelectronics, and energy harvesting. However, not many sensor applications have been attempted or demonstrated with proper characteristics, especially for DNA or genome sequencing purposes.

### Silicon Layers for Sensor Bridges

In various embodiments, a biosensor herein may comprise a nanobridge made of a silicon material rather than a TMD material. As used herein, “Si-material” or “Si-type” bridge, nanobridge or layer may comprise pure crystalline silicon or any type of doped silicon semiconductor material, as described herein.

In various embodiments, a silicon nanobridge may comprise a semiconductor wire or a ribbon structure. In various embodiments, a silicon nanobridge may comprise material derived from a silicon-on-insulator (SOI) wafer, and may take the form of a nanostructure or related semiconductor nanoribbon or nanowire structure.

A single nanobridge structure of crystalline silicon may be obtained from silicon-on-insulator (SOI) semiconductor substrate (or other related semiconductor layers), which can be made into a reliable nanobridge sensor structure since the material is already properly-doped semiconductor films with such a nanobridge material being less sensitive to nanopatterning-related damages. Therefore, a need for high temperature processing is minimized. Unique SOI-based fabrication of nanobridge structures, processing methods and applications of such a single-bridge SOI-based nanoribbon biosensors are disclosed in the drawing figures.

The thickness of a doped SOI Si thin film nanobridge is desirably thin, e.g., <30 nm, preferably <20 nm thick, more preferably <10 nm thick layer, and even more preferably <5 nm, with a width of <40 nm, preferably <20 nm, more preferably <10 nm, and even more preferably <3 nm. A smaller cross-section nanowire or nanoribbon bridge across two mating electrodes can provide higher signal to noise ratio during sequencing interactions.

Also, a smaller cross-section nanowire or nanoribbon bridge of SOI-based Si nanobridge or crystalline semiconductors in general (e.g., <10 nm width, preferably <5 nm width, more preferably 3 nm width), ensures that only a single (or at most a few) nanobridge structures will attach to form one or at most a few bridges, due to size exclusion, instead of multiple bridges, with the latter introducing multiple, undesirably complicated signals from the parallel bridges stuck together on the same electrode lead pair. A single and narrower sensor bridge will also prefer a single-molecule polymerase placement for each electrode pair.

Substantially narrowed SOI-Si structure for a single nanobridge can be prepared using highly advanced nanofabrication of Si and SiO₂ structures. Another approach to further ensure a single bridge formation of SOI-Si, according to various aspects of the present disclosure, is to utilize a nano-mask array, for example, by placing an array of e.g., <5 nm wide graphene, carbon, ceramic or metallic nanoribbon mask so that the SOI-Si underneath can be patterned and shaped to become narrow nanobridges. Such pre-made narrowed graphene, carbon, ceramic or metallic nanoribbon mask can be prepared on another substrate followed by stamp transfer using a PMMA (Polymethyl methacrylate), PDMS (Polydimethylsiloxane) or other polymer type stamp materials. Such pre-made narrowed bridge can be used as a sensor bridge on which a single enzyme molecule such as polymerase can be attached.

In various embodiments, such SOI-Si type or other semiconductor ribbon strips can be split into two nanobridges between which another molecular bridge (such as a single DNA or peptide) can be attached for ease of polymerase bonding and nucleotide analysis. Such splitting of amorphous semiconductor into two separated parts with a nanogap in between (e.g., 20-100 nm) can be accomplished by e.g., focused laser beam slicing, focused ion beam cutting or patterning and etching. The ends of the split ribbons facing each other can desirably be sharpened to a pointed-tip geometry of e.g., 2-5 nm radius of curvature, so as to facilitate an attachment of a single DNA or a single peptide molecular bridge, using either electric field alignment, flow alignment in a microfluidic chamber, or stamp transfer of pre-aligned DNA or peptide. Stamp transfer of pre-aligned DNA or peptide array can be made using PMMA, PDMS or other polymer type soft stamps.

### Biosensors

Disclosed herein are label-free DNA or RNA sequencing device structures utilizing a TMD- or Si-based frame with an enzyme polymerase for detection of electronic signals when an individual nucleotide is attached onto a nucleic acid template. In various embodiments, two dimensional semiconductors of processed, defective or nanoporous Transition Metal Dichalcogenide (TMD) layer material are employed so as to utilize altered bandgaps of the TMD layer and enhanced attachment of single biomolecules. In various embodiments, the TMD-based sequencing systems disclosed herein can be assembled into a massively parallel configuration for rapid analysis of targets including nucleotides, in particular for applications of sequencing of a DNA molecule, or a collection of such molecules constituting an entire genome. Such systems are also useful for DNA-based information storage, for which the writing is performed by encoding specific nucleotide-based arrangements or sequences and the reading is carried out by sequencing analysis using TMD-bridge based molecular sensor array.

A bridge-configured sensor structure comprising an elongated nano-dimension, crystalline semiconductor wire or ribbon, such as silicon or doped silicon, is another way of producing label-free molecular sensors for genome sequencing without introducing complicated fluorescence imaging methodologies. Such semiconductor nanowires of, e.g., made of Si derived from silicon-on-insulator (SOI) wafer, can be connected to a pair of electrodes (with optional gate structure) in high density electronic circuit assembly, also equipped with microfluidic environment comprising floating DNAs, nucleotides, enzyme polymerase molecules, etc. It is possible to attach a polymerase single molecule (or few molecules) to such a nanobridge or elongated biomolecule bridge, using, for example, functionalities and ligands such as biotin-streptavidin, antibody-antigen, pyrene or peptide complexes.

Inorganic nanobridges, either van der Waals force connected or metallization connected to the device electrode leads can offer much higher electrical conductivity and substantially higher electrical sensor signals than organic nanobridges as the use of undesirably high electrical resistance ligands or attaching functionalities can be minimized.

In various embodiments of the present disclosure, biosensor bridges connected between a pair of conducting electrodes (such as made of Au, Ag, Pd, Pt, Ru, Rh, Al, Cu, Ni, etc.), can be accomplished by an active attachment route using electrical field (electrophoretic alignment and attachment using various AC or DC mode of electric fields, often at a level of 0.2 to 10 volts). Alternatively, aligned nanobridge structures connecting the mating electrodes can also be achieved by stamp transfer of pre-aligned (or pre-patterned) inorganic nanobridge array from another substrate using PMMA, PDMS or other stamp materials. On the device substrate or temporary preparation substrate, the nanobridge array in parallelly aligned configuration is prepared either by nanopatterning by lithographic means, flow alignment in a microfluidic chamber, or by electrical field alignment (e.g., dielectrophoretic alignment using AC or DC electric field).

While a number of nanomaterials can be considered for such nanobridge structures, these nanomaterials need to be processed into nano-dimension wires or ribbons by nanolithography or other treatments, which tends to cause damage to crystallographic structures or disruption of atomic arrangements causing unintended changes or deterioration of physical or electronic properties. While a high temperature annealing process (e.g., 500° to 1000° C.) can sometimes repair or reduce such damages, the use of high temperature processing steps often damages electronic device structure and hence should be avoided for ease of device manufacturing and reliability.

For accurate signal detection on nucleotide attachment events (or other biomolecule attachment events, e.g., to polymerase, to enable electrical signal detection for genome sequencing), it is highly desirable to provide a single elongated bridge (made of inorganic or organic nanowires or nanoribbons) between mating electrodes (made of Au, Pd or other conducting lead wires). If multiple nanobridges are attached between the two mating electrodes, often clumped together, multiple signals may occur by the presence of parallel sensors, which makes the analysis of such complicated signals very difficult.

### Information Storage

DNA data storage is a process of digital encoding and decoding binary data, to and from synthesized or duplicated DNA strands. For example, the binary code information storage of (00), (01), (10) and (11) can be replaced by various arrangements of oligonucleotides (A, C, G, T). DNA molecules are genetic blueprints for living organisms, and the information stored in DNA is known to last more than 10,000 years under certain environment. With its huge capacity (many orders of magnitude larger than what is possible with current technology) to store enormous amount of information in very small space, DNA storage could be the answer to a modern era problem of too much information that needs to stored, e.g., on the order of hundreds of zettabytes every year in the near future. Currently available information storage capability including magnetic disk, tape, optical or other related technologies can cover only a fraction of such a needed capacity.

While substantial progress has been made in DNA information storage in recent years, cost effective data storage techniques for practical applications are yet to be achieved. For efficient retrieval of stored information, the encoded DNA nucleotide arrangements need to be decoded by, e.g., reading (or sequence analysis). A fast, economic method of reading the encoded DNA information is essential for the success of DNA based data storage. This disclosure also provides new methods and device structure to enable such progress.

Biosensors having a TMD or silicon nanobridge provide reliable DNA genome analysis performance and are more easily amenable to scalable manufacturing, because the need for high temperature processing is minimized. The disclosed structures herein are also useful for DNA-based large-capacity information storage devices including archival or randomly-accessible-memory and logic devices.

### TMD Compositions

In various embodiments, a sequencing device is provided comprising: (a) an electrode array of conducting electrode pairs, each pair of electrodes comprising a source and a drain electrode separated by a nanogap, said electrode array deposited and patterned on a dielectric substrate, and optionally comprising a third electrode as a gated system; (b) a single-layer or few-layer thick transition metal dichalcogenide (TMD) layer disposed on each pair of electrodes, connecting each source and drain electrode within each pair, and bridging each nanogap of each pair; (c) a dielectric masking layer disposed on the TMD layer and comprising size-limited openings that define exposed TMD regions, each opening sized so as to allow only a single enzyme biomolecule to fit therein and to attach onto the exposed TMD region defined by each opening; (d) an enzyme molecule attached to each exposed TMD region such that only one enzyme molecule is found within each opening; and (d) a microfluidic system encasing the electrode array, wherein attachment or detachment of a biomolecule selected from the group consisting of a nucleotide monomer, a protein, and a DNA segment, onto the enzyme molecule, one at a time, can be monitored as a uniquely identifiable electrical signal pulse to determine the specific nature of the biomolecule attaching or detaching.

In various embodiments, the dielectric substrate comprises SiO₂. In various embodiments, the dielectric substrate comprises SiO₂ or Al₂O₃.

In various embodiments, the TMD is selected from MoS₂, WS₂, TiS₂, ZrS₂, HfS₂, VS₂, NbS₂, TaS₂, TcS₂, ReS₂, CoS₂, RhS₂, IrS₂, NiS₂, PdS₂, PtS₂ and their modifications or combinations. In various embodiments, the TMD is MoS₂. In various embodiments, the TMD is WS₂.

In various embodiments, the TMD is selected from MoS₂, WS₂, TiS₂, ZrS₂, HfS₂, VS₂, NbS₂, TaS₂, TcS₂, ReS₂, CoS₂, RhS₂, IrS₂, NiS₂, PdS₂, PtS₂ and their modifications or combinations, including modified stoichiometry of sulfur contents having MX₍₂₋ₓ₎ or MX₍₂₊ₓ₎ wherein x is in the range of 0-1.0, preferably in the range of 0-0.5, even more preferably 0-0.3. In various embodiments, the TMD is selected from MoS₂, WS₂, TiS₂, ZrS₂, HfS₂, VS₂, NbS₂, TaS₂, TcS₂, ReS₂, CoS₂, RhS₂, IrS₂, NiS₂, PdS₂, PtS₂ and their modifications or combinations and the stoichiometry of sulfur is not modified.

In various embodiments, the TMD is selected from MoSe₂, WSe₂, or TiSe₂, ZrSe₂, HfSe₂, VSe₂, NbSe₂, TaSe₂, TcSe₂, ReSe₂, CoSe₂, RhSe₂, IrSe₂, NiSe₂, PdSe₂, PtSe₂ and their modifications or combinations, including modified stoichiometry of selenium contents having MX₍₂₋ₓ₎ or MX₍₂₊ₓ₎ with the desired value of x in the range of 0-1.0, preferably in the range of 0-0.5, even more preferably 0-0.3. In various embodiments, the TMD is selected from MoSe₂, WSe₂, or TiSe₂, ZrSe₂, HfSe₂, VSe₂, NbSe₂, TaSe₂, TcSe₂, ReSe₂, CoSe₂, RhSe₂, IrSe₂, NiSe₂, PdSe₂, PtSe₂ and their modifications or combinations and the stoichiometry of selenium is not modified.

In various embodiments, defects are artificially introduced into TMD. In various embodiments, the defects are introduced to increase bandgap. In various embodiments, the defects are introduced to provide active site edge locations for strong adhesion of bridge structures or biomolecules such as enzyme molecules.

In various embodiments, TMD is selected from MoTe₂, WTe₂, or TiTe₂, ZrTe₂, HfTe₂, VTe₂, NbTe₂, TaTe₂, TcTe₂, ReTe₂, CoTe₂, RhTe₂, IrTe₂, NiTe₂, PdTe₂, PtTe₂ and their modifications or combinations.

In various embodiments, TMD is selected from MoTe₂, WTe₂, or TiTe₂, ZrTe₂, Hffe₂, VTe₂, NbTe₂, TaTe₂, TcTe₂, ReTe₂, CoTe₂, RhTe₂, IrTe₂, NiTe₂, PdTe₂, PtTe₂ and their modifications or combinations, including modified stoichiometry of Tellurium contents having MX₍₂₋ₓ₎ or MX₍₂₊ₓ₎ with the desired value of x in the range of 0-1.0, preferably in the range of 0-0.5, even more preferably 0-0.3. In various embodiments, TMD is selected from MoTe₂, WTe₂, or TiTe₂, ZrTe₂, Hffe₂, VTe₂, NbTe₂, TaTe₂, TcTe₂, ReTe₂, CoTe₂, RhTe₂, IrTe₂, NiTe₂, PdTe₂, PtTe₂ and their modifications or combinations and the stoichiometry of tellurium is not modified.

In various embodiments, the tellurium stoichiometry is intentionally altered so as to provide vacancy defects, interstitial defects, and aggregated defects in order to increase the surface energy of the TMD layer and enhance the adhesion of biomolecule to the bridge sensor for stronger sensor signals.

In various embodiments, the TMD comprises a mixed TMD selected from TMD compounds in which the MX2 compound has mixed metals and/or mixed chalcogenide, selected from the group consisting of Mo(SₓSe_{y}Te_{z})₂, W(SₓSe_{y}Te_{z})₂, Ti(SₓSe_{y}Te_{z})₂, Zr(SₓSe_{y}Te_{z})₂, Hf(SₓSe_{y}Te_{z})₂, V(SₓSe_{y}Te_{z})₂, Nb(SₓSe_{y}Te_{z})₂, Ta(SₓSe_{y}Te_{z})₂, Tc(SₓSe_{y}Te_{z})₂, Re(SₓSe_{y}Te_{z})₂, Co(SₓSe_{y}Te_{z})₂, Rh(SₓSe_{y}Te_{z})₂, Ir(SₓSe_{y}Te_{z})₂, Ni(SₓSe_{y}Te_{z})₂, Pd(SₓSe_{y}Te_{z})₂, and Pt(SₓSe_{y}Te_{z})₂ wherein the combined (x+y+z) is 1-3, 0.5-1.5, or 0.7-1.3.

In various embodiments, two or more metals are combined for sulfur containing, Se-containing or Te-containing TMD layers.

In various embodiments, the TMD layer comprises (MoₓW_{y}Co_{z})S₂ or (HfₓW_{y}Co_{z})Te₂.

In various embodiments, the TMD comprises a M_{(1-w)}N_{y}X_{(2-z)}Y_{z} structure in which the transition metal M is partially substituted with non-transition elements N, with a concentration of w and the N element selected from one or more of Al, Si, Ga, Ge, In, Sn, Sb, Bi, Al, Na, K, Ca, Mg, Sr, Ba, with the w value in the range of 0-0.3, and the chalcogenide element X partially substituted with a non-chalcogenide element Y, with the Y element selected from one or more of Li, B, C, N, O, P, F, Cl, I, with the z value in the range of 0-0.3. In various embodiments, the w value is greater than 0.3, for example, greater than 0.4, greater than 0.5, greater than 0.6, greater than 0.7, greater than 0.8, greater than 0.9, or greater than 1.0. In various embodiments, the z value is greater than 0.3, for example, greater than 0.4, greater than 0.5, greater than 0.6, greater than 0.7, greater than 0.8, greater than 0.9, or greater than 1.0.

### Silicon Compositions

In various embodiments, a sequencing device is provided comprising: (a) an electrode array of conducting electrode pairs, each pair of electrodes comprising a source and a drain electrode separated by a nanogap, said electrode array deposited and patterned on a dielectric substrate, and optionally comprising a third electrode as a gated system; (b) a single-layer or few-layer thick silicon or doped silicon semiconductor layer disposed on each pair of electrodes, connecting each source and drain electrode within each pair, and bridging each nanogap of each pair; (c) a dielectric masking layer disposed on the silicon layer and comprising size-limited openings that define exposed silicon regions, each opening sized so as to allow only a single enzyme biomolecule to fit therein and to attach onto the exposed silicon region defined by each opening; (d) an enzyme molecule attached to each exposed silicon region such that only one enzyme molecule is found within each opening; and (d) a microfluidic system encasing the electrode array, wherein attachment or detachment of a biomolecule selected from the group consisting of a nucleotide monomer, a protein, and a DNA segment, onto the enzyme molecule, one at a time, can be monitored as a uniquely identifiable electrical signal pulse to determine the specific nature of the biomolecule attaching or detaching.

In various embodiments, the electrodes in each electrode pair may comprise Au, Ag, Pd, Pt, Ru, Rh, Al, Cu, Ni, etc.

In various embodiments, the dielectric substrate comprises SiO₂. In various embodiments, the dielectric substrate comprises SiO₂ or Al₂O₃.

In various embodiments, the silicon material forming the nanobridge comprises pure crystalline silicon, such as single crystalline silicon.

In various embodiments, the silicon material forming the nanobridge comprises an n- or p-doped silicon semiconductor material. Dopants for n-type or p-type doped silicon semiconductor may comprise acceptors from Group III elements or donors from Group V elements. Dopants for n- or p-doped silicon semiconductor include, but are not limited to As, B, P, Sb, Ga, Zn, and Fe.

In various embodiments, a silicon nanobridge may be obtained from a SOI wafer manufactured with the desired silicon material (e.g., single crystalline silicon, n-type silicon semiconductor, or p-type silicon semiconductor).

Various aspects of the present disclosure provide biosensor structures, materials and geometries, as well as fabrication methods and application methods, such as described below in reference to the various drawing figures.

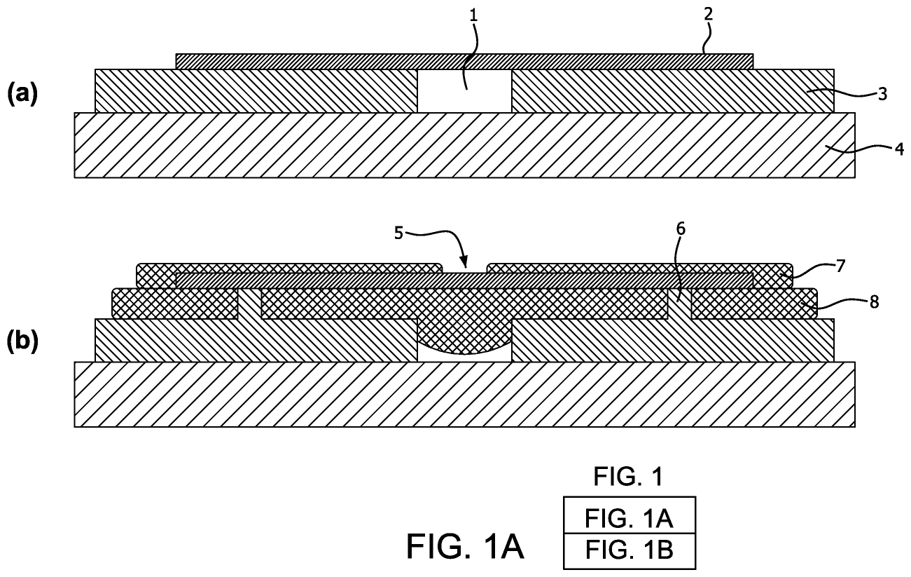

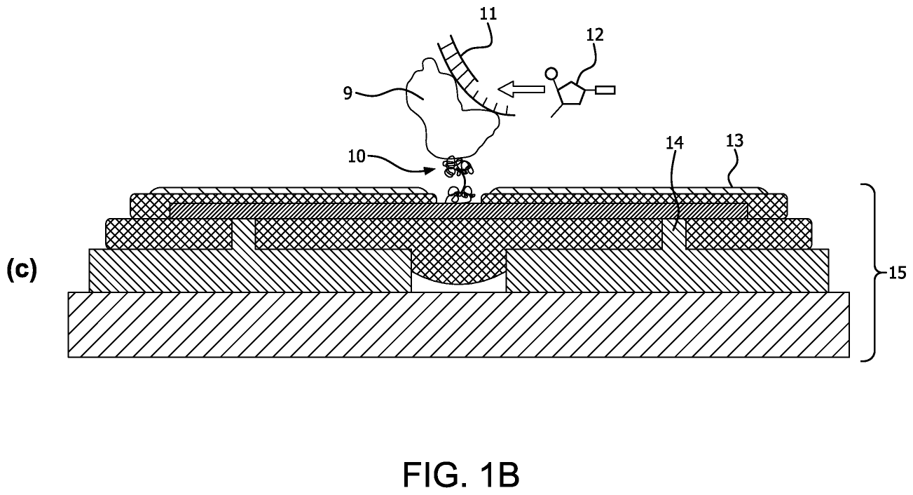

FIGS. 1A-1B illustrate various sensor structures and manufacturing steps for making a complete sensor in accordance with various aspects of the present disclosure. In general, structure (a) shows a nanoribbon over a nanogap; structure (b) shows a nanoribbon on a planarize surface; and structure (c) shows a size limited nanoribbon wherein a dielectric coating obscures most of the nanoribbon, leaving only a small exposed region of nanoribbon that fits only a single biomolecule, like an enzyme molecule.

FIG. 1A(a) shows a conducting electrode pair 3 disposed on a dielectric substrate 4. There may be a plurality of pairs of electrodes, such as an array; however for clarity only the cross-section of one pair of electrodes is shown. The electrodes 3 may comprise conducting metals or alloys, such as Au, Ag, Pd, Pt, Ru, Rh, Al, Cu, Ni, etc., or their various alloys. The spacing 1 between electrodes is referred to as the nanogap 1, which may be dimensioned from about 2 nm to 100 nm, or 2 nm to about 20 nm, and preferably from about 5 nm to about 20 nm. The underlying substrate 4 may comprise such materials as SiO₂, or SiO₂ on Si. FIG. 1A(a) also shows a suspended TMD (e.g., MoS₂) or Si-material nanoribbon 2 (or other semiconductor nanobridge), having a rectangular, or narrow-ribbon configuration. Such a layer may be nanopatterned by nanoimprinting or e-beam lithography, or applied onto the electrodes and over each nanogap by a stamp transfer process, as explained below. The TMD material may comprise any transition metal dichalcogenide as disclosed herein. The Si-material for the Si nanoribbon may comprise pure single crystal silicon or any p- or n-type doped silicon semiconductor, such as obtained from an SOI wafer.

FIG. 1A(b) shows a device structure comprising conducting pillars 6, that in various aspects function as vertical extensions of the underlying electrodes. The pillars may comprise the same material as the electrodes, such as the same metal, or the pillars may comprise deposits of a different material. As shown, the device structure (b) comprises a size-limited (e.g., 5 nm dia.), exposed MoS₂ or other TMD or Si-material region 5, appropriately sized to accommodate only a single biomolecule (e.g., a polymerase enzyme) for binding to the exposed portion of the nanobridge. That is, the opening 5 is dimensioned so as to allow only a single biomolecule, such as a single polymerase enzyme, to contact and attach to the exposed TMD or Si-material bridge. The coating of the majority of the TMD or Si-material bridge, leaving only the opening 5, may be accomplished by a size-limiting dielectric coating 7 (PMMA or SiO₂) disposed over the TMD or Si-material bridge 2. Alternatively, a pre-planarized PMMA layer 8 provides a flat surface for nanoimprinting or other uses. The nanopillars 6 may comprise vertical Au nanopillars (about 5 nm-50 nm diameter), or may comprise other metals, such as Pd, Pt, Ru, Rh, etc. In various embodiments, the pillars may comprise island structures rather than cylinders, e.g., island Au pads, like hemispheres or other structures having at least a pillar top surface region.

In various embodiments, the metallic conducting electrode pair is selected from Au, Ag, Pd, Pt, Ru, Rh, Al, Cu, Ni, or their alloys. In various embodiments, the conducting electrode pair 3 may comprise Al, Cu, Ru, Pt or Pd, whereas the pillars 6 may comprise Ru, Pt, or Pd.

In various embodiments, the nanogap 1 is from about 5 nm to about 20 nm. In various embodiments, the nanogap is less than 5 nm, for example less than 3 nm, or less than 1.0 nm. In various embodiments, the nanogap is greater than 20 nm, for example, greater than 25 nm, greater than 30 nm, greater than 35 nm, greater than 40 nm, greater than 45 nm, or greater than 50 nm.

In various embodiments, the size-limiting openings 5 are preferably less than 30 nm average equivalent diameter each, more preferably less than 20 nm equivalent diameter, even more preferably less than 10 nm equivalent diameter. The openings 5 can be fashioned by lithographic or nanofabrication defined coverage of a dielectric material layer, polymer or ceramic in all the areas outside a specific, size-limited region intended for attaching only a single molecule. The term “average equivalent diameter” is used when the openings 5 are not circular, but where the calculated surface area can be expressed as though the openings are circular. Polymerase molecules, as well as streptavidin-type linked molecules, often have a steric size on the order of ˜5 nm regime. With the openings 5 thus configured, only one biomolecule fits within each opening to attach to the exposed TMD or Si-material inside the opening.

FIG. 1B(c) shows a MoS₂-based or Si-based, two electrode or gated molecular sensor 15 for genome or DNA sequencing. Such a sensor is configured to detect a change or perturbation in an electrical parameter (e.g., a current pulse) upon the attachment of a nucleotide or other biomolecules to the polymerase 9. In other configurations, the detection event may comprise protein sensing. The MoS₂-or Si-based, two electrode or gated molecular sensor for genome or DNA sequencing 15 is shown to include an optional blocking agent 13 (e.g., PEG, Teflon, etc.) to prevent biomolecule adhesion, and vertical Au or other conducting metal nanopillars 14. The MoS₂- or Si-based, two electrode or gated molecular sensor for genome or DNA sequencing 15 is shown to include a single enzyme molecule 9 (e.g., DNA or RNA polymerase, or other sensors) attached onto a size limited region of the MoS₂ or Si-material bridge, with a biotin-streptavidin complex 10 (with another connecting moiety, e.g., a silane). The sensor 15 is shown with a double-stranded DNA molecule 11 attached to the enzyme 9. Nucleotide monomers 12 (e.g., A, T, C, G) are detected as they participate in the polymerase reaction.

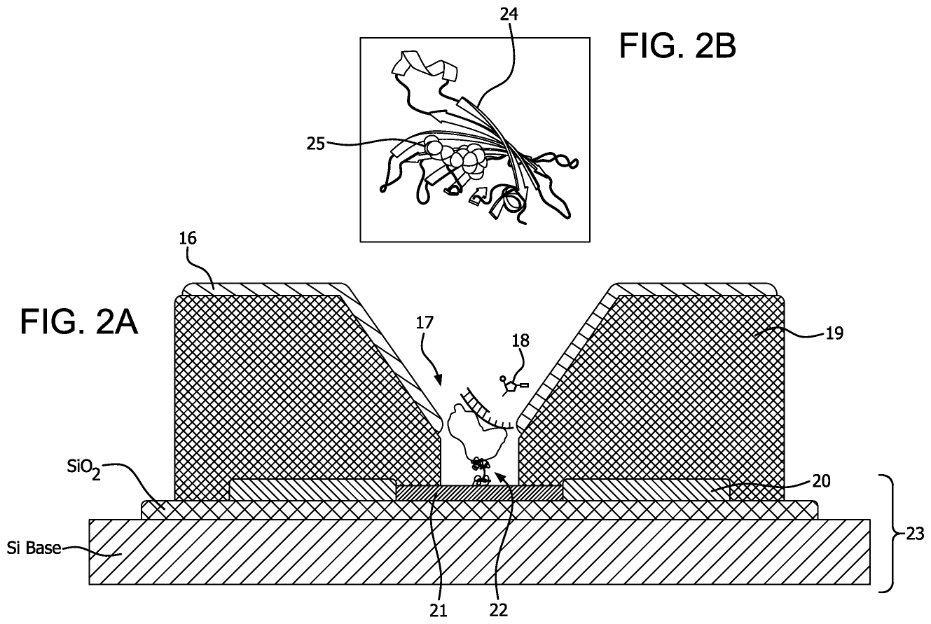

FIGS. 2A and 2B show Design #2 architecture for “Geometrically-guided, single DNA polymerase” molecular sensor on MoS₂ or Si-material bridge (with the exposed area of the TMD or Si-nanoribbon bridge at the base of the channel size restricted to ˜5 nm dia., which is comparable in size to a streptavidin or polymerase molecule). FIG. 2A shows a FET sensor 23 based on TMD (MoS₂ or WS₂) or Si-type nanoribbon bridge for genome or DNA sequencing via detection of change of current pulse upon attaching of nucleotide or other biomolecules. The structure 23 includes a PMMA, HSQ or silica based funnel structure 19 (i.e., a guiding structure), which comprises a tapered channel prepared by e-beam or microfabrication, or by nanoimprint mold (Si or metal). The structure 23 includes a deposited metal electrode pair 20 for source and drain, with an optional third electrode as a gate structure (not illustrated), optional blocking agent 16 (e.g., PEG, Teflon) to prevent unwanted biomolecule adhesion), MoS₂ or Si-material nanoribbon bridge 21, a single polymerase 17 attached onto a size-limited MoS₂ or Si-material nanoribbon bridge, and biotin (on a semiconductor nanoribbon surface, optionally with silane linker) plus streptavidin (on polymerase), or alternatively, an antibody-antigen linkage, or other binding mechanism 22 to connect the polymerase or other enzyme to the TMD or Si-material layer 21. Nucleotide monomer 18 (e.g., A, T, C, G, etc.), fragments, or proteins are to be detected upon polymerase reaction. FIG. 2B shows a polymerase binding mechanism comprising streptavidin 24 and biotin 25.

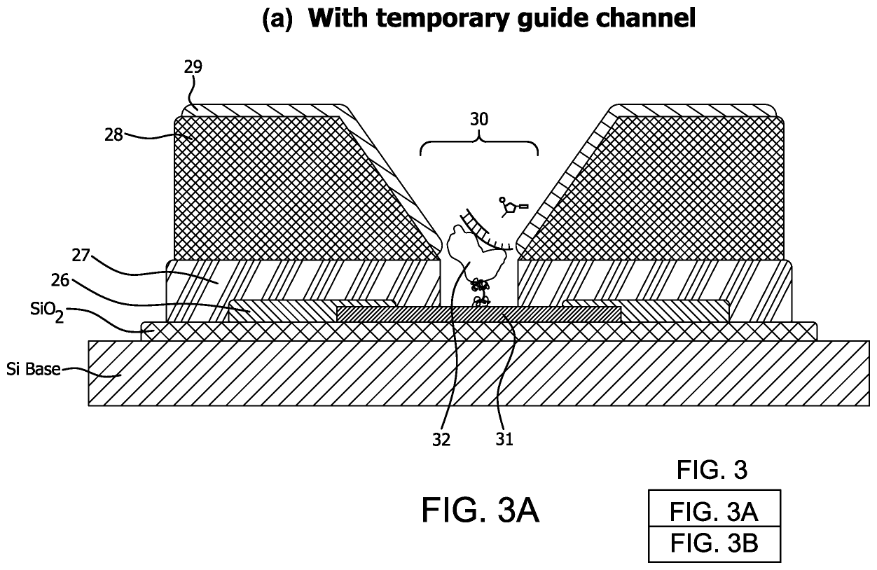

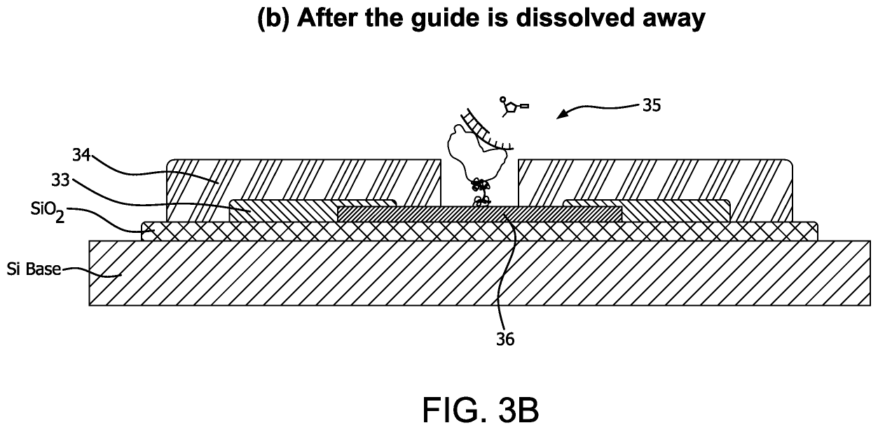

FIGS. 3A and 3B show Design #3 architecture for “Temporary guiding channel to construct single DNA polymerase” molecular sensor on TMD (e.g., MoS₂) or Si-material bridge (with the exposed area at the base of the channel size restricted to ˜5 nm dia., which is comparable in size to a streptavidin or polymerase molecule).

FIG. 3A(a) shows a structure with a temporary guide channel (or guidance channel) 30 in place. The structure optionally includes a temporary and removable (sacrificial) layer 28 (e.g., dextrin, polysaccharide, polyvinyl alcohol, etc.) that can be dissolved in water, alcohol or various other solvents, or dissolvable resist such as PMMA, with optional blocking agent 29 (e.g., PEG, Teflon); an insoluble dielectric 27; deposited metal electrode pair 26 for source and drain, with an optional third electrode as a gate electrode (not illustrated); and TMD (e.g., MoS₂) or Si-type nanoribbon bridge 31. A single polymerase 32 is shown attached to the TMD or Si-material bridge 31.

FIG. 3B(b) shows the device structure after the temporary guide channel is dissolved away. The structure includes a dielectric layer 34 such as SiO₂, cured PMMA, HSQ, etc. This layer is previously disposed completely over the first and second electrodes 33 to entirely encase them, but only partially disposed over the TMD, Si, or doped Si-semiconductor layer 36 so as to leave an opening vertically through the dielectric layer to an exposed portion of the TMD, Si, or doped Si-semiconductor layer. As shown in FIG. 3B(b), the exposed portion has a diameter less than the width of the nanogap between the previously deposited metal electrodes 33. In this way, the nanogap between electrodes 33 does not need to be precisely manufactured since it will be the dielectric layer 34 that determines the size of the opening to accommodate the biomolecule. As shown, the structure includes a single polymerase sensor complex 35 partially hidden below the dielectric layer 34, attached to the TMD, Si, or doped Si-semiconductor layer 36 by such linkers as streptavidin-biotin linkage.

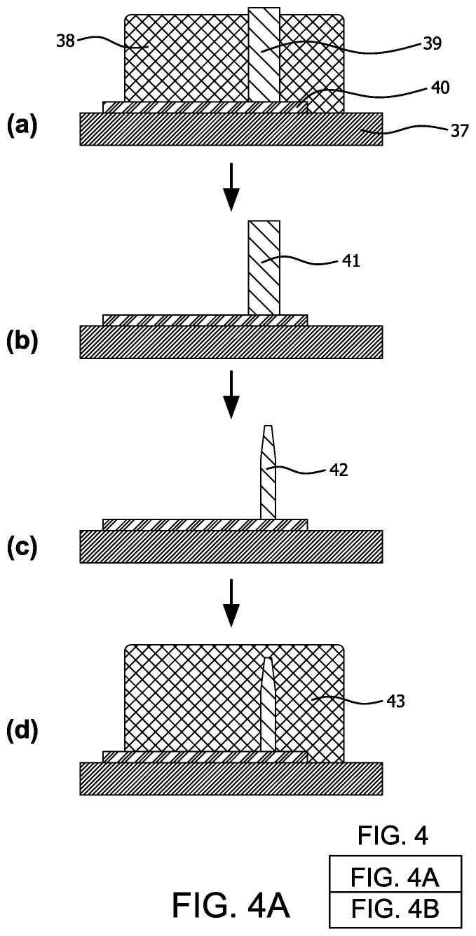

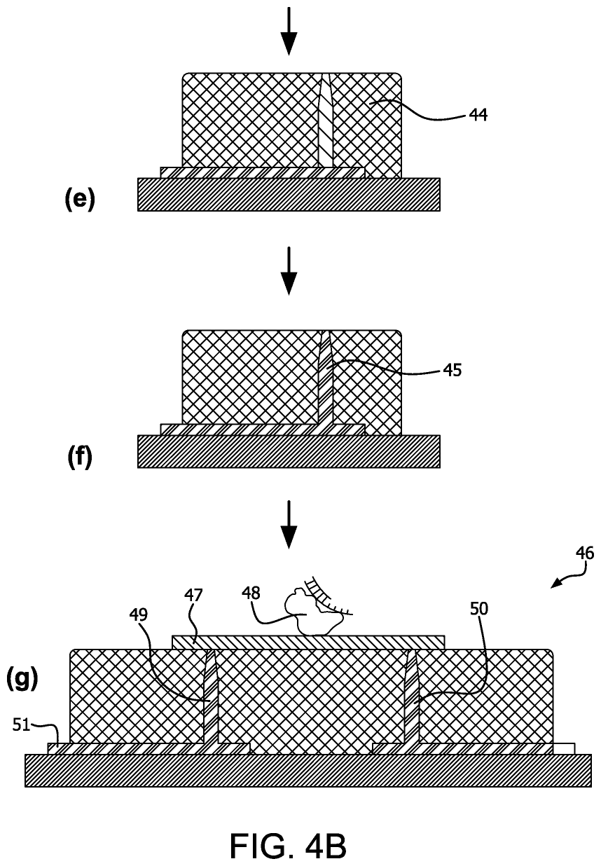

FIGS. 4A and 4B show a fabrication method designed to provide narrowed pillar structures that then can be used in a sensor device. In FIG. 4A(a), the structure includes Au or other conducting lead wire 40 on a substrate 37. E-beam or nanoimprint lithography may be used to configure a vertical hole in the PMMA 38 down to the surface of the conducting layer 40. The resulting hole is then filled with Cu or Ni, such as by electroless or electrodeposition, or a sputter and lift-off process, to produce a Cu or Ni nanopillar 39. In FIG. 4A(b), the PMMA is dissolved away to expose the bare Cu or Ni nanopillar 41 (which may be about 20-50 nm in dia.). In FIG. 4A(c), chemical or RIE etch is used on the Cu or Ni nanopillar to form a nanowire 42 with reduced diameter and tapered tip (e.g., with a tip ending in a diameter of ˜5 nm). In FIG. 4A(d), PMMA 43 is cast over the Cu or Ni nanowire 42 to cover the diameter-reduced Cu or Ni nanowire. In FIG. 4B(e), the PMMA 44 is planarized in height by RIE to expose the Cu or Ni nanopillar top. In FIG. 4B(f), the Cu or Ni nanopillar is dissolved away to create a hole, and Au or other metal is electrodeposited to form the new pillar 45 (having only ˜5 nm dia. tip area). This narrowed nanopillar 45 can then size-limit the MoS₂ or Si-material width or the number of any attachable nanowire/nano-ribbon bridge. FIG. 4B(g) shows a complete molecular bridge solid state sensor 46 that is configured to detect a change of current, such as a perturbation or pulse, or other signals upon attaching or detaching of nucleotides or other biomolecules. Molecular bridge solid state sensor 46 includes: Au or other conducting lead wire 51 disposed on the underlying substrate; Au nanopillars 49 and 50; MoS₂ or Si-type nanobridge 47; and single enzyme (e.g., DNA or RNA polymerase) 48 attached onto the MoS₂ or Si-material nanobridge 47. In this way, the TMD or Si-material nanobridge 47 is elevated above the gap between electrodes 51 by the height of the pillars 49/50.

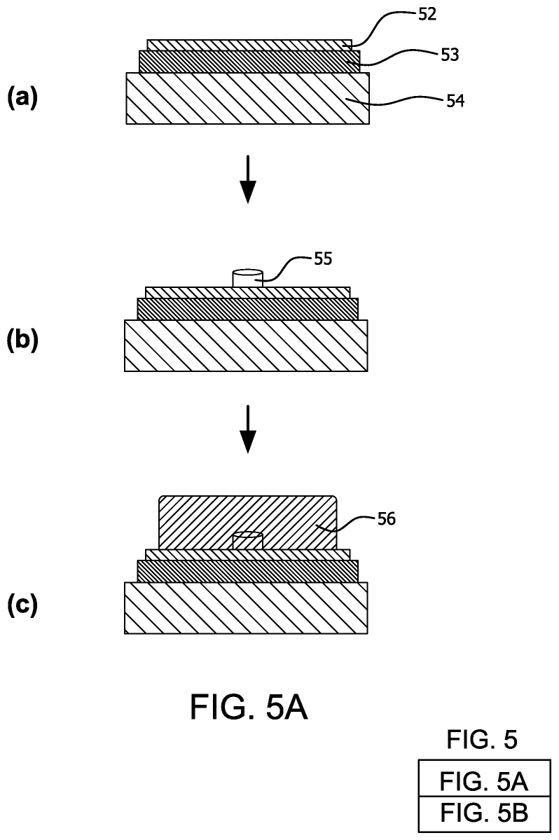

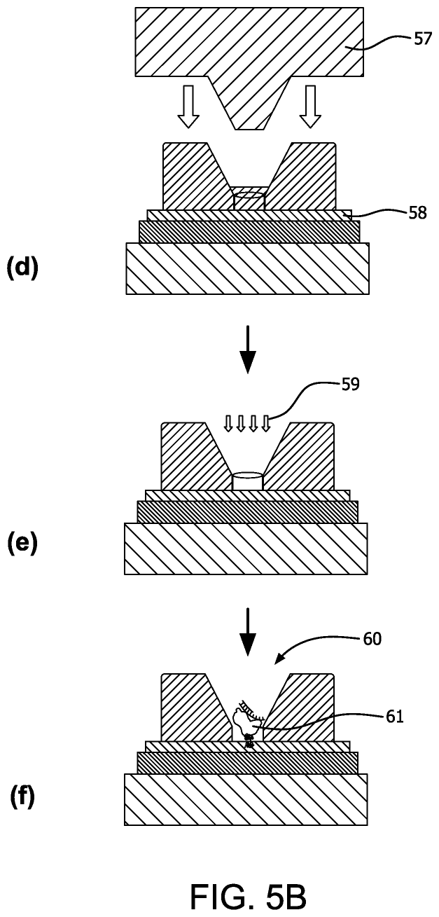

FIGS. 5A and 5B show another sensor fabrication method. In FIG. 5A(a), the structure (a) comprises a MoS₂ or Si-material nanoribbon 52 disposed on an underlayer 53 (e.g., PMMA, SiO₂), which is disposed on a substrate 54 (e.g., Si). In FIG. 5A(b), a dissolvable pillar 55 (5-10 nm dia., comprising Cu, Ni, etc.) is deposited by e-beam or NIL lithography onto the TMD or Si-material nanoribbon. In FIG. 5A(c), a resist 56 (e.g., PMMA, HSQ, or another type of silica-containing resist) is spin coated over the entirety of the pillar 55. In FIG. 5B(d), an imprint is made with a die 57 (e.g., the die 57 made of Si, SiO₂, or metal alloy mold). The MoS₂ or Si-material nanoribbon 58 remains unchanged throughout the process. In FIG. 5B(e), the metal pillar is exposed by RIE etching 59 (the directional arrows indicating the etching process to clean off the pillar). In FIG. 5B(f), the metal pillar is dissolved away to create a funnel shaped guiding structure 60 with e.g., a 5-10 nm hole, so that only a single streptavidin molecule or only a single polymerase or other enzyme can be placed therein for more accurate sequencing by the finished device. The structure in FIG. 5B(f) includes a single polymerase 61 attached onto a size-limited MoS₂ or Si-material bridge (by e.g., by biotin-streptavidin linkage or other linkage).

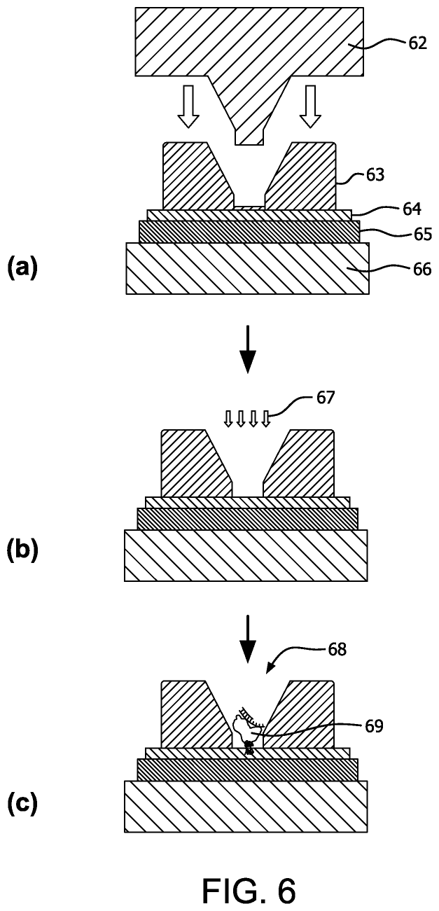

FIG. 6 shows another method of fabricating a sensor device. In FIG. 6(a), the structure (a) comprises a MoS₂ or Si-material nanoribbon 64 on a PMMA layer 65, which is on a Si substrate 66. A PMMA or HSQ (which can be later converted to SiO₂) thermo-plastic resist or other silica-containing resist 63 is imprinted with an appropriately shaped die 62 (e.g., made of Si, SiO₂, or metal alloy mold) having a <10 nm dia. tip protrusion as illustrated. In FIG. 6(b), RIE etch 67 exposes the base MoS₂ or Si-material bridge nanoribbon surface. In FIG. 6(c), through the funnel shaped guiding structure 68 with e.g., 5-10 nm hole is placed only a single streptavidin or only a single polymerase or other enzyme for more accurate sequencing from the finished sensor device. FIG. 6(c) shows a single polymerase 69 attached onto a size-limited MoS₂ or Si-material bridge (by e.g., a biotin-streptavidin linkage or other linkage).

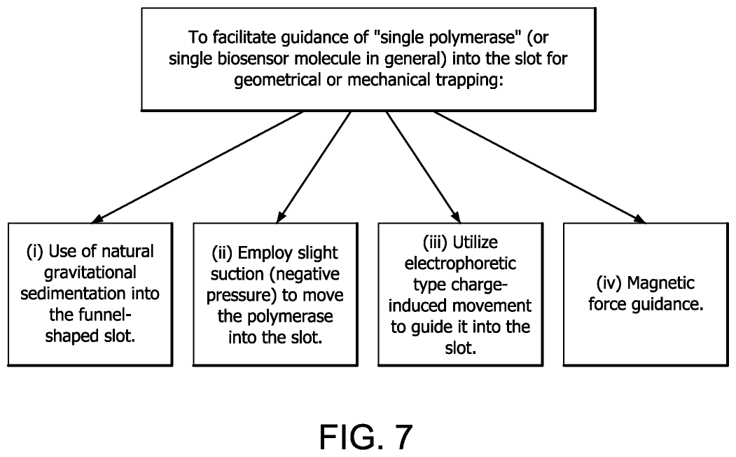

FIG. 7 shows how to facilitate guidance of “single polymerase” (or a single biosensor molecule in general) into the slot for geometrical or mechanical trapping: (i) use of natural gravitational sedimentation into the funnel-shaped slot; (ii) employment of a slight suction (negative pressure) to move the polymerase into the slot; (iii) utilizing electrophoretic type charge-induced movement to guide the molecule into the slot; or (iv) magnetic force for guidance.

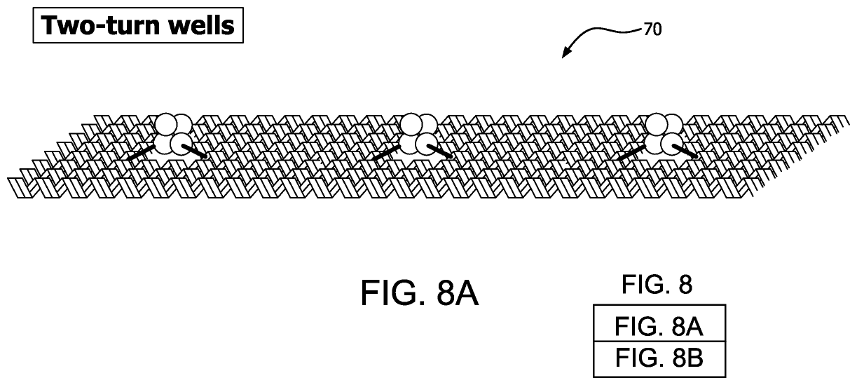

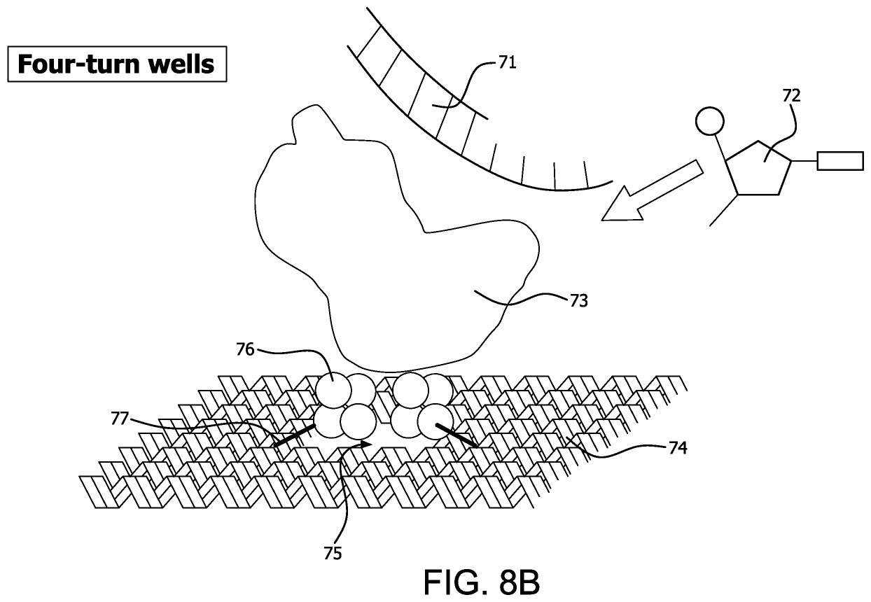

FIGS. 8A and 8B show Design #4 “Use of size-confined DNA assembly well”: size-confined nano-well by DNA assembly on MoS₂ or Si-type nanoribbon bridge; periodic array of streptavidin (SA) on electrode surface, or such a template further processed/coated (with dielectric or conductive layer) for geometrical size-limiting constraint to enable only (or essentially only) a single enzyme molecule attachment; SA nanoarray is prepared by size-selective capturing of a single SA tetramer in a two-turn well (FIGS. 8A, 70) or capturing of two tetramers trapped in a four-turn well (FIG. 8B).

As illustrated in FIG. 8A, two-turn wells 70 (20 nm-10 μm) spaced apart are used for each electrode pair in a multi-sensor array. In FIG. 8B, a four-turn well 75 (˜7×10 nm well) in a DNA bundle 74 has trapped streptavidin 76 (two streptavidin tetramers), and biotin 77 linking. FIG. 8B shows double strand DNA 71, nucleotide monomer 72 (A, T, C, G, etc.), short fragments, proteins to be detected, and preferably a single enzyme molecule 73 (e.g., DNA or RNA polymerase) attached onto size limited TMD bridge like MoS₂ or WS₂ or a Si-material bridge.

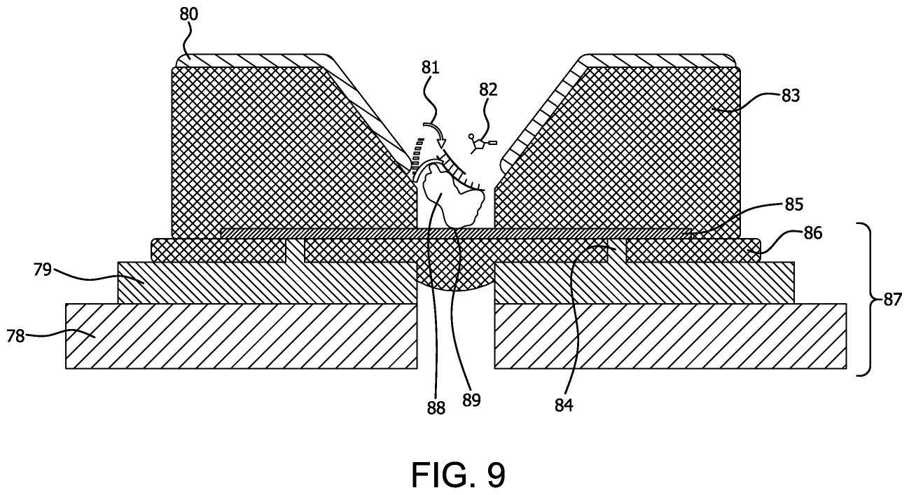

FIG. 9 shows Design #5 “Use of physically-trapped single polymerase on TMD (MoS₂) or Si-material bridge on vertical nanopillar conductor.” Such a device design reduces contact resistance and further increases the FET signals, (e.g., by at least 10 times), and eliminates the need for biotin and streptavidin type linkages since the enzyme may be guided into the device to attach directly to the TMD, Si- or Si-semiconductor layer 85. The sensor device uses shape memory polymer or an alloy structure to physically force electrical contact (Van der Waals force bond) of a polymerase molecule onto the TMD (MoS₂) or Si-type bridge. In FIG. 9, the molecular sensor on the TMD or Si-material bridge 87, usable for genome or DNA sequencing via detection of change of current pulse upon attaching of nucleotide or other biomolecules, comprises Au or other conducting metal leads 79 disposed on a dielectric substrate 78 (SiO₂, etc., on Si). Electrically connected to the metal leads 79 are vertical Au (or other conducting metal) nanopillars 84, which can be formed, for example, by the method embodied in FIGS. 4A-4B and discussed above. In this structure, the PMMA or other dielectric material layer 86 is planarized such that the top surface of the PMMA layer 86 is entirely planar with the exposed tips of the nanopillars 84. In this way, the exposed tops of the nanopillars 84 will appear as discs. The planarized PMMA 86 layer enables deposition or transfer of the TMD, Si- or Si-semiconductor layer 85 on the device with guaranteed contact to the tops of the two nanopillars 84. The PMMA channel 83 is used to direct the enzyme or enzyme complex into the exposed region of the TMD, Si- or Si-semiconductor layer 85.

In FIG. 9, the conducting electrodes 79 are separated by a nanogap having a width W1. This width may be from about 2 nm to about 100 nm, and more preferably about 5 nm to about 20 nm. The pair of nanopillars 84 are separated from one another by a width W2, wherein in various embodiments W2≥W1. In various embodiments, W2 may be from about 5 nm to about 100 nm. With the TMD, Si- or Si-semiconductor layer 85 attached across and connecting to the tops of the nanopillars 84, the TMD, Si- or Si-semiconductor layer 85 is suspended over the space between the nanopillars 84 and the over the nanogap. Since the former space is filled in with dielectric material 86, that material may partially or entirely fill in the nanogap between the electrodes 79. As shown in the example of FIG. 9, the dielectric material entirely fills in the space between the nanopillars 84 but only partially fills in the nanogap.

In FIG. 9, blocking agent 80 (e.g., PEG, Teflon) may be used to prevent unwanted biomolecule adhesion to areas other than the exposed portion of the TMD or Si-material bridge 85 below. Shape memory change 81 enables fixing a single polymerase 88 directly to this size-limited TMD or Si-material bridge, with nucleotide monomer 82 (e.g., A, T, C, G, U, etc.) to be detected on polymerase reaction. In FIG. 9, the linkage 89 between enzyme 89 and TMD or Si-material bridge 85 may comprise biotin-streptavidin, or an antibody-antigen linkage, or other linkage mechanism, or the connection may comprise a bond between a modified amino acid of the enzyme and the transition metal or silicon of layer 85.

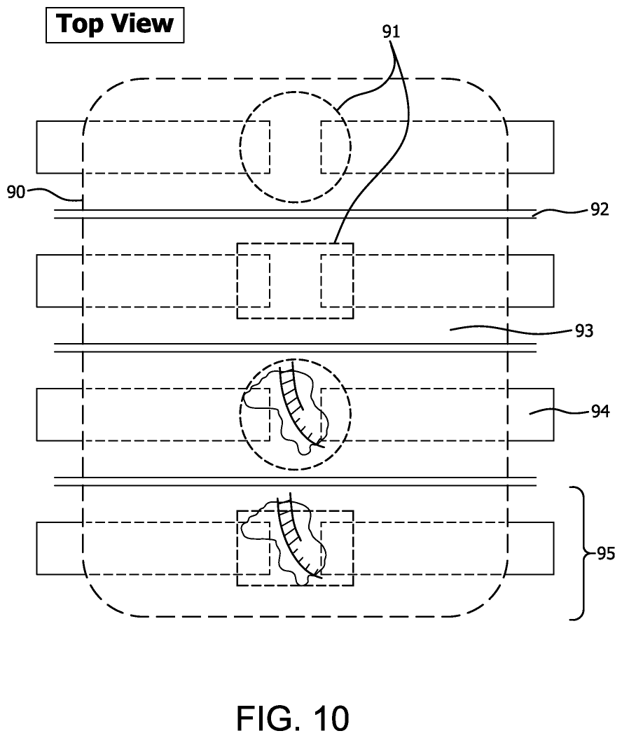

With reference to FIG. 10, a MoS₂ or Si-material sheet 90 is configured with size-limited (circular, square, or other desired shapes) areas 91 to locally and selectively expose the TMD or Si-material. A large sheet can be placed by lifting up of floating sheet onto device surface covering many electrode pairs lithography-patterned, FIB separated, or mask patterned. Optional FIB (focused ion beam) slicing, e-beam/NIL slicing 92 of MoS₂ or Si-material can be used to separate portions from adjacent devices. The exposed island region 91 is surrounded by pattern defined dielectric coating mask 93 (e.g., PMMA, PDMS, other adhesion blocking PEG type layer or Teflon, etc.) to prevent/minimize biomolecule attachment. An array of conducting electrode and lead wires 94 are used for signal detection. In FIG. 10, one complete molecular bridge sensor 95 is illustrated and comprises a MoS₂, WS₂ or Si-material layer bridge.

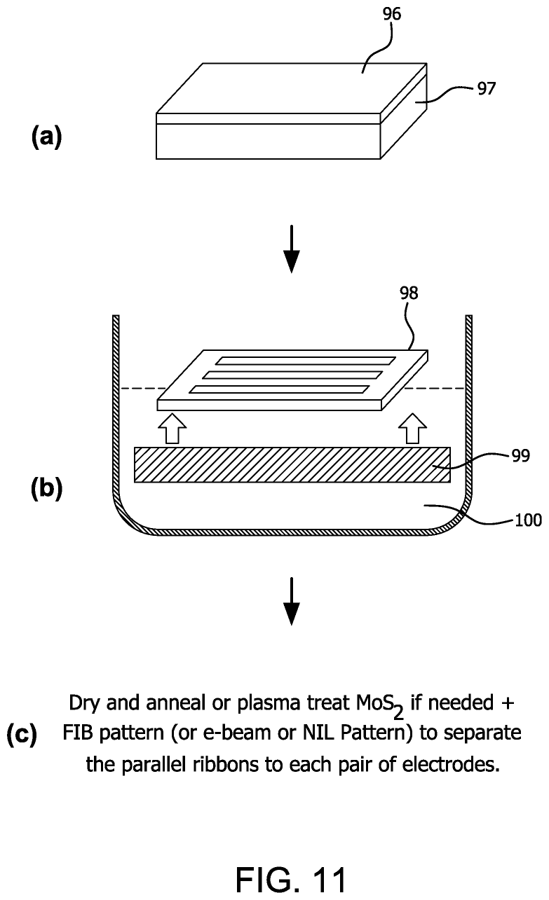

FIG. 11 shows a “Wet transfer of un-patterned or pre-patterned MoS₂ ribbon array: MoS₂ films are synthetized at >700° C., so they are preferably pre-made and then transferred onto a sequencing device. The next steps in the process are to (i) release the ribbon array in an aqueous solution (chemically dissolve away the substrate); (ii) place the Au electrode device (wafer) in the solution; (iii) lift up the wafer to catch the floating MoS₂ film; (iv) dry and anneal the wafer to strongly bond the MoS₂ film (or nano-ribbon) onto the electrode surface; and (v) prepare size-limited MoS₂ regions and attach DNA polymerase. The finished sensor functions by generating detectable sequence signals corresponding to nucleotide attachment to the polymerase.

In FIG. 11(a), the substructure (a) may be prepared by synthesis of TMD 96 like MoS₂ or WS₂ (e.g., by exfoliation, CVD or sulfurization) on Si or metal substrate 97. Substrate 97 can comprise Si with SiO₂, a SOI wafer or a metal layer. In the transition from (a) to (b), substrate is etched away for floating MoS₂.

In FIG. 11(b), floating MoS₂ or WS₂ 98 (no pattern or pre-pattered into parallel ribbons, still attached) in H₂O or alcohol 100, with SiO₂-coated Si substrate 99 (optionally with Au electrode pair disposed on it), is lifted up to pick up the MoS₂ layer.

In FIG. 11(c), the wafer is dried and annealed or plasma treated if needed, and optionally FIB patterned (or e-beam or NIL patterned) to separate the parallel ribbons such that one ribbon is associated to each pair of electrodes.

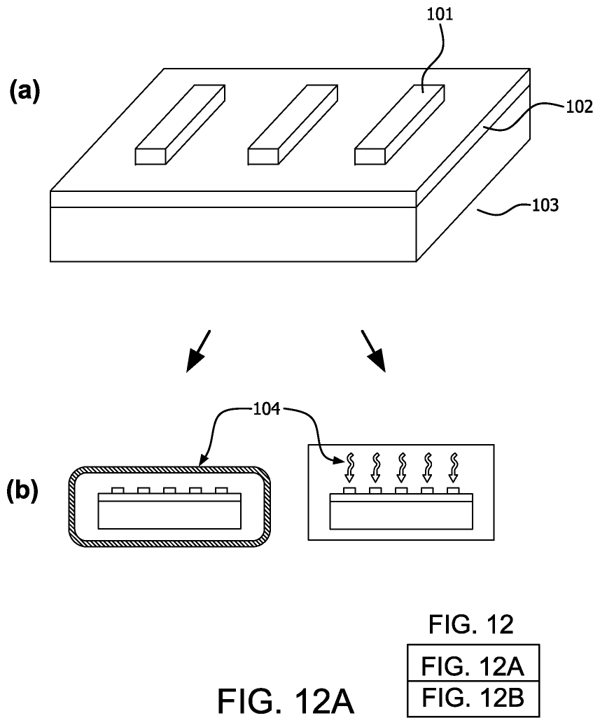

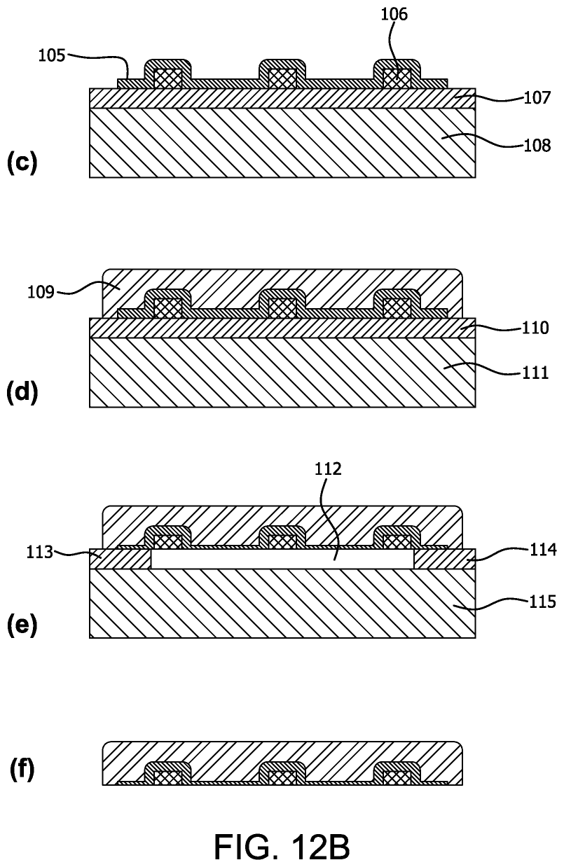

FIGS. 12A and 12B illustrate various aspects of a fabrication method for parallel nanoribbons. The structure in FIG. 12A(a) comprises a nano-patterned MoS₂ or Si-material nanoribbon array 101 disposed on SiO₂ 102, which is disposed on Si base 103.

In FIG. 12A(b), high-temp annealing or plasma treatment 104 can be used to repair any possible damages on the patterning.

In FIG. 12B(c), nano-patterned MoS₂ or Si-material nanoribbon array 106 on SiO₂ 107 on Si base 108 is optionally coated with a removable polymer 105 (e.g., dextrin, glucose, grease, wax).

In FIG. 12B(d), the nano-patterned MoS₂ or Si-material nanoribbon array on SiO₂ 110 on Si base 111, optionally coated with removable polymer (e.g., dextrin, glucose, grease, wax), is then cast over with a PMMA or PDMS elastomer 109, and then cured.

In FIG. 12B(e), the SiO₂ 112 underneath is etched away through intentionally added slots in the PMMA or PDMS to release the potted nanoribbon array. This can also be done in two steps of PMMA followed by PDMS. In FIG. 12B(e), the structure shown comprises portions of SiO₂ 113 and 114 after etching, on the Si base 115.

FIG. 12B(f) shows the released material that can be washed lightly to make the MoS₂ or Si-material nanoribbons protrude slightly, dried, and then transferred and pressed onto the sequencing device to be released, and form a non-bridge between two mating metallic electrodes.

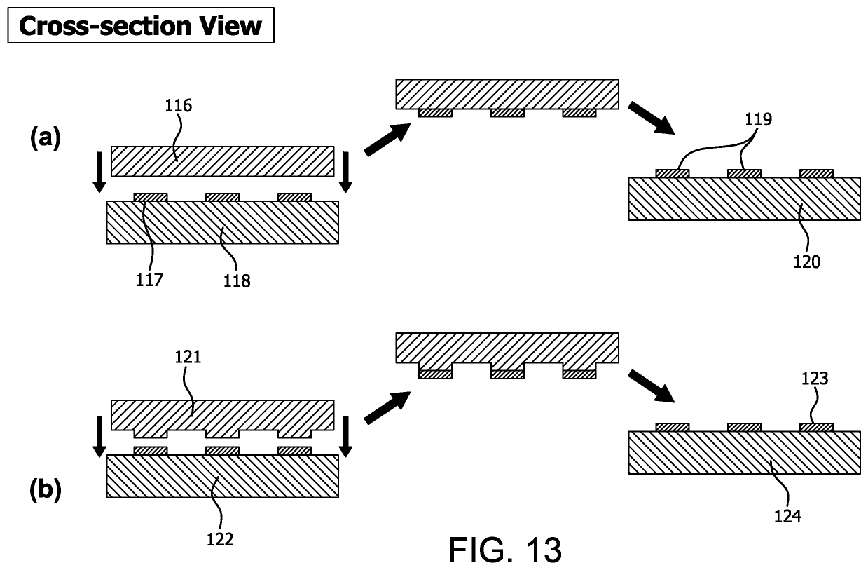

FIG. 13 shows another fabrication method in accordance with the present disclosure. FIG. 13(a) shows a PDMS stamp 116 (with a flat contact surface), and MoS₂ nano-ribbon array 117 (nanopatterned on a flat substrate 118). The transformation illustrated comprises a pickup of the nanoribbons by stamp, and a release of the nano-ribbons 119 on a device surface 120 (e.g., as a bridge between two mating electrodes). In FIG. 13(b) a pre-shaped PDMS stamp 121 is used to pick up the nanoribbons from substrate 122 by stamp, and then the nanoribbons 123 are released on the device surface 124 (e.g., as a bridge between two mating electrodes).

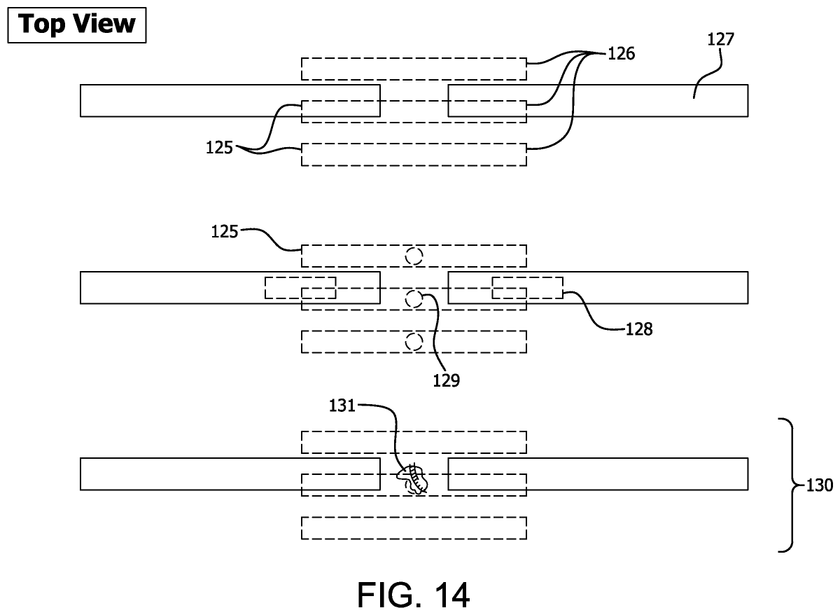

FIG. 14 shows MoS₂ or Si-material nanoribbons 125 in a pre-patterned parallel array 126 of MoS₂ or Si or doped silicon with redundant ribbon array (made by e.g., e-beam lithography, nanoimprint lithography, template-assisted nanopatterning, etc.). The imprint may be transferred onto an electrode array so as to increase the probability of MoS₂ or Si-bridge connection. The MoS₂ or other TMD or Si-material ribbons can be Van der Waals force attached, or optionally metallization deposit (metallization deposit 128 (Ti, Au, Ni, etc.) over MoS₂ or Si-material can be made to more firmly attach the ribbons on the electrodes. In FIG. 14, optional addition of local biotin-binding-enhancing coating 129, or surrounding-area-masking can be employed to limit the exposed MoS₂ or Si-material regions to ˜5-10 nm dia. One complete molecular sensor 130 is illustrated that comprises a MoS₂ or Si-material bridge with a polymerase assembly, shown with a polymerase enzyme 131 (with associated biotin-streptavidin or other type linkage).

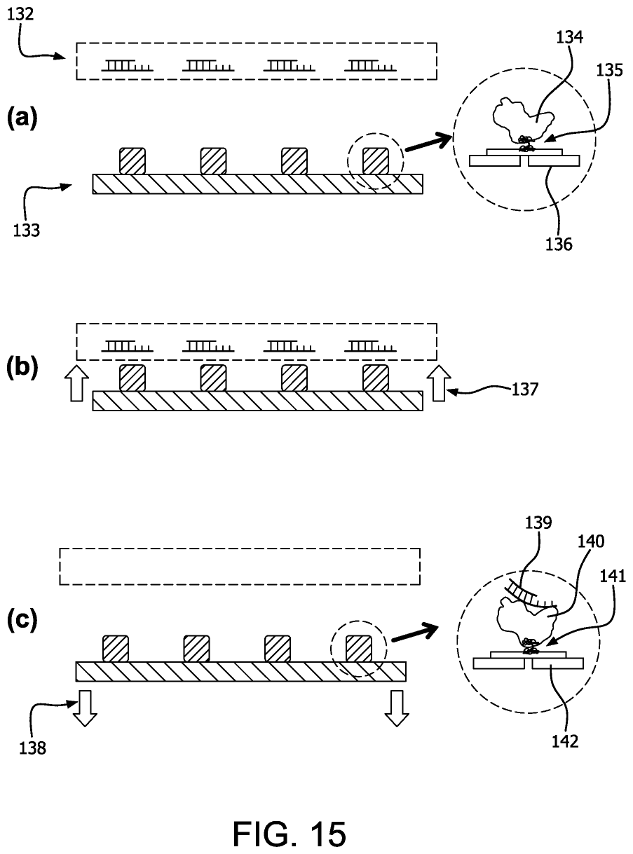

FIG. 15 shows another fabrication method in accordance with the present disclosure. In FIG. 15(a), a tethered array of encoded (memory written) DNA fragments 132 is periodically positioned on a substrate. Molecular nanobridges (MoS₂ or Si-material) polymerase sensor array 133 (w/o DNA template). Magnified view shows polymerase 134, linker 135, and Au electrode pair 136. In FIG. 15(b), polymerase-MoS₂ or Si-type nanobridge array approaching DNA array being released (indicated by 137). In FIG. 15(c), nanobridge sensor array polymerase molecule array picks up DNA templates and is moved away (indicated by 138). Magnified view shows DNA template 139, polymerase 140, linker 141 and Au electrode 142.

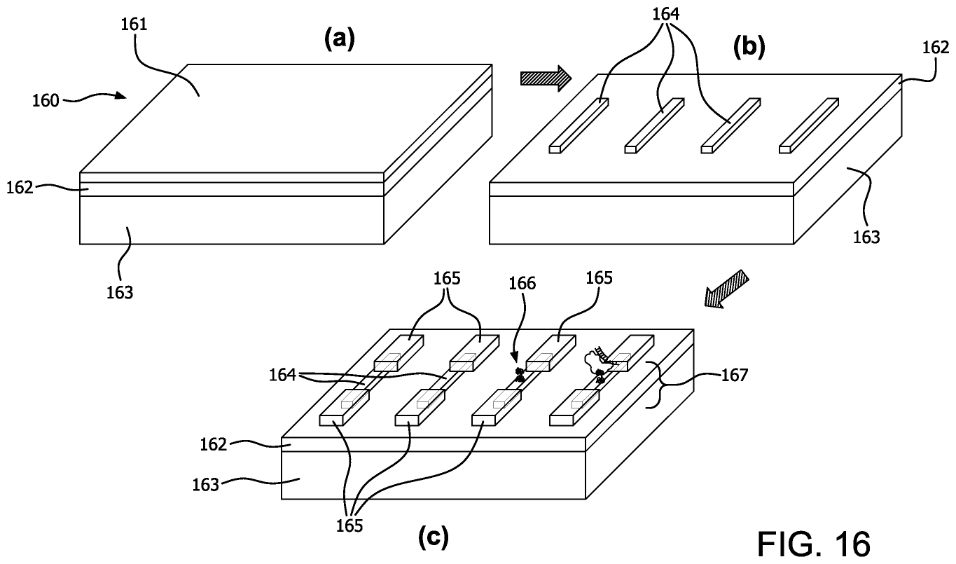

FIG. 16 illustrates a fabrication method in accordance with various embodiments of the present disclosure. The method begins with a silicon-on-insulator (SOI) wafer 160, shown in (a), comprising a Si-material layer 161 disposed on a dielectric substrate layer 162, e.g., SiO₂, which is disposed on the Si base 163 of the SOI wafer. As discussed throughout, the Si-material top layer may comprise crystalline Si, such as single crystal Si, or a p-type or n-type doped Si. This Si material layer 161 may have a thickness of less than about 50 nm. In various embodiments, the thickness of the Si-material layer 161 is less than about 20 nm. The Si-material layer may comprise various n-doped or p-doped semiconductor material, such as Si, Ga-As, In-P, ZnO based, or other thin layer semiconductor materials such as graphene bases, 2D semiconductor-based (e.g., MoS₂, WS₂ or other chalcogenide based semiconductors. While doped semiconductors are preferred, if the nanoribbon width is narrowed sufficiently per the patterning described below, there is a possibility of bandgap opening, so undoped or minimally-doped semiconductors may also be utilized. Such SOI wafers are commercially available with the type of semiconductor material 161 as desired.

With continued reference to FIG. 16, the next step in the fabrication is to pattern the Si-material layer 161 into a pattern of parallel Si-material nanoribbons 164. This patterning may be accomplished by e-beam lithography, nanoimprint lithography, graphoepitaxy, shadow-mask RIE etching, or by various other methods of patterning. The resulting structure (b) comprises parallel Si-material nanoribbons disposed on the dielectric substrate layer 162 of the SOI wafer. The nanoribbons 164 can of course by patterned into more of rod shapes, or other cross-sectional shapes as needed for particular sensor structures. In various embodiments, the width of each ribbon or rod is less than about 30 nm, preferably less than about 10 nm, and most preferably less than about 6 nm. In an alternative to manipulating an SOI wafer, an array of parallel semiconductor nanoribbons or rods can be fabricated on a different substrate surface, and then taken off the surface (e.g., using PDMS or adhesive polymer stamps) and transferred onto a device surface. Some adhesion-enhancing layers that can be etch-removed later (e.g., Au, Ag, Cu, Ni, etc.) can be optionally added as the surface contacting the semiconductor nanoribbon to pick them up more easily for transfer onto the device substrate surface.

With reference now to structure (c) in FIG. 16, pairs of electrodes 165 may be deposited onto the array of parallel nanoribbons 164 such that two electrodes in any single pair of electrodes contacts either side of a single nanoribbon as illustrated. A partially finished sensor unit is illustrated as the second assembly from the right of the structure, comprising a biotin-streptavidin complex 166, with optional linker such as a silane, attached to the nanoribbon 164. A finished sensor unit is shown at the far right of the structure, comprising a sensor complex 167 further comprising a single molecule enzyme, such as a DNA or RNA polymerase, attached to the Si-material nanoribbon bridge, with a DNA or RNA template attached and ready for nucleotide interaction and detection. The wafer structure can be encased in a fluidic chamber such that the appropriate biomolecules can be circulated over the exposed nanoribbons for attachment onto the nanoribbons. A complete sensor having the sensor complex works by providing changes in an electrical property of the Si nanoribbon FET bridge, such as current perturbations, when nucleotides attach to the polymerase and participate in polymerase reactions.

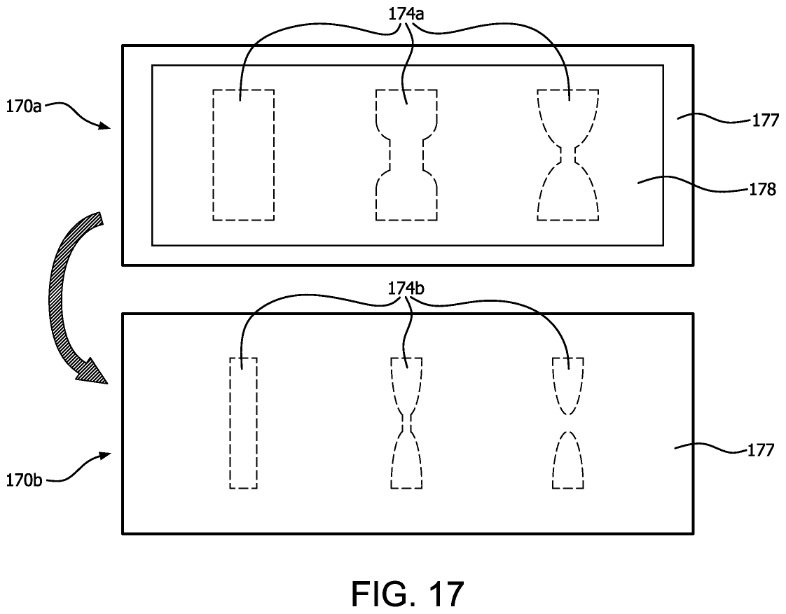

FIG. 17 illustrates various dimensions and shapes for semiconductor nanobridges, which are controllable by nanofabrication, nanoimprinting, shadow-mask RIE etch or by repeated oxidation/chemical etch. In structure 170a at the top of FIG. 17, different shaped and/or sized nanoimprinted, nanopatterned, shadow-mask RIE etched Si-material, (Si or a doped Si semiconductor), TMD material such as MoS₂, graphene or an amorphous semiconductor nanoribbons 174a, are disposed on a thin Si semiconductor or n- or p-type Si semiconductor layer 178, which is disposed on the substrate 177 of an SOI wafer. Alternatively, the structure of layers 178 and 177 may be another type of dielectric covered substrate instead of an SOI wafer. Sturcutre 170a is converted to structure 170b by further narrowing the Si or other semiconductor nanoribbon array. This width reduction can be accomplished by nanopatterning or repeated oxidation and chemical etching. The resulting width-reduced nanoribbons 174b are left disposed on the underlying SOI wafer substrate 177. Having reduced nanoribbon width (i) opens a bandgap; (ii) allows higher signal-to-noise ratio electrical signals; and (iii) enables a single or just a few streptavidin or DNA molecules to attach to any single nanobridge surface. If a nanoribbon tip is sharpened such that the nanoribbon separates into portions, as per the nanoribbon illustrated at the far right, electric field concentration of the sharp-tip electrode can allow a single DNA or other type of biomolecular bridge to form by dielectrophoretic (DEP) type or electrical field alignment. This sharpening of a nanobridge as shown to produce a two-portion semiconductor can be accomplished by further etching or by FIB slicing.

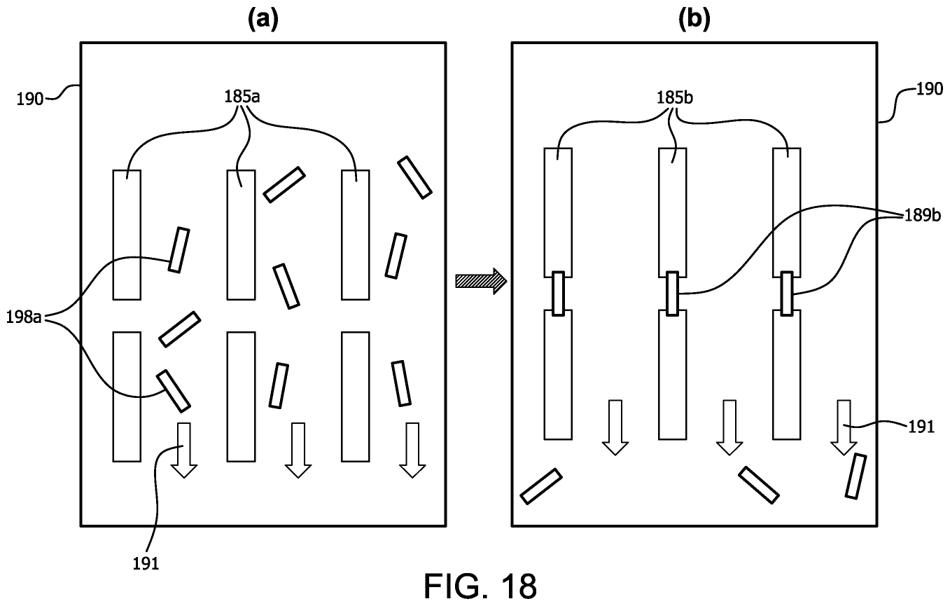

FIG. 18 illustrates a method of alignment and attachment of floating nanoribbons 198a onto electrode pairs 185a, such as arranged in an array of electrode pairs. The structure (a) is before any electric field is applied and structure (b) is after electric field alignment of the nanobridges to the electrode pairs. In this method, the floating nanoribbons 198a may comprise Si nanoribbons, graphene, metal dichalcogenide, such as MoS₂ type nanoribbons, amorphous semiconductor nanoribbons, any of which may be pre-patterned or pre-narrowed. For the method, the electrode pairs are encased in a fluidic chamber 190, like a flow cell. The block arrows 191 indicate the flow of the microfluidic solution moving. To attach the nanoribbons to the electrode pairs, dielectrophoretic (DEP) alignment is used, whereby an AC or DC electric field is applied to the electrode pair array. The applied AC field may be in the range of about 100 mV to about 20 V, and preferably from about 50 mV to about 5V, with a frequency of about 50 Hz to about 10 MHz, and preferably from about 100 Hz to about 100 KHz. The result after electric field alignment is shown in structure (b), wherein nanobridges 189b are now attached to electrode pairs 185b, with one nanobridge per electrode pair. As illustrated, there is an excess of nanobridges and thus some do not participate in attachment.

### Additional Considerations

In various embodiments, a single-molecule biosensor comprises: a conducting electrode pair disposed on a substrate, the electrode pair comprising a first electrode and a second electrode spaced-apart from the first electrode by a nanogap; a contiguous transition metal dichalcogenide (TMD) layer disposed on the first electrode and on the second electrode, the TMD layer configured as a bridge suspended over the nanogap; an enzyme molecule attached to a region of the TMD layer suspended over the nanogap; and a microfluidic system encasing the conducting electrode pair, the contiguous TMD layer and the enzyme molecule attached thereto.

In various embodiments, the TMD layer comprises a TMD selected from the group consisting of MoS₂, WS₂, TiS₂, ZrS₂, HfS₂, VS₂, NbS₂, TaS₂, TcS₂, ReS₂, CoS₂, RhS₂, IrS₂, NiS₂, PdS₂, PtS₂, and mixtures thereof.

In various embodiments, the TMD layer comprises a TMD selected from the group consisting of MoSe₂, WSe₂, TiSe₂, ZrSe₂, HfSe₂, VSe₂, NbSe₂, TaSe₂, TcSe₂, ReSe₂, CoSe₂, RhSe₂, IrSe₂, NiSe₂, PdSe₂, PtSe₂, and mixtures thereof.

In various embodiments, the TMD layer comprises at least one TMD of structure MSe₍₂₋ₓ₎ or MSe₍₂₊ₓ₎, wherein x is 0-0.3, and M is Mo, W, Ti, Zr, Hf, V, Nb, Ta, Tc, Re, Co, Rh, Ir, Ni, Pd, or Pt.

In various embodiments, the TMD layer comprises a TMD selected from the group consisting of MoTe₂, WTe₂, TiTe₂, ZrTe₂, HfTe₂, VTe₂, NbTe₂, TaTe₂, TcTe₂, ReTe₂, CoTe₂, RhTe₂, IrTe₂, NiTe₂, PdTe₂, PtTe₂, and mixtures thereof.

In various embodiments, the TMD layer comprises a mixed TMD compound selected from the group consisting of Mo(SₓSe_{y}Te_{z})₂, W(SₓSe_{y}Te_{z})₂, Ti(SₓSe_{y}Te_{z})₂, Zr(SₓSe_{y}Te_{z})₂, Hf(SₓSe_{y}Te_{z})₂, V(SₓSe_{y}Te_{z})₂, Nb(SₓSe_{y}Te_{z})₂, Ta(SₓSe_{y}Te_{z})₂, Tc(SₓSe_{y}Te_{z})₂, Re(SₓSe_{y}Te_{z})₂, Co(SₓSe_{y}Te_{z})₂, Rh(SₓSe_{y}Te_{z})₂, Ir(SₓSe_{y}Te_{z})₂, Ni(SₓSe_{y}Te_{z})₂, Pd(SₓSe_{y}Te_{z})₂, Pt(SₓSe_{y}Te_{z})₂, and mixtures thereof, wherein (x+y+z) is 0.7-1.3.

In various embodiments, the TMD layer comprises at least one TMD compound of structure M_{(1-w)}N_{y}X_{(2-z)}Y_{z}, wherein M is Al, Si, Ga, Ge, In, Sn, Sb, Bi, Na, K, Ca, Mg, Sr, or Ba; X is S, Se, or Te; Y is Li, B, C, N, O, P, F, Cl, or I; w is 0-0.3; and z is 0-0.3.

In various embodiments, the substrate comprises SiO₂ or Al₂O₃.

In various embodiments, the pair of conducting electrodes comprise at least one of Au, Pt, Ag, Pd, Rh, Ru, or alloys thereof.

In various embodiments, the nanogap is from about 1 nm to about 50 nm.

In various embodiments, the single-molecule biosensor further comprises a dielectric, ceramic or polymer coating layer disposed on the TMD layer on a side opposite the first and second electrodes, wherein the dielectric, ceramic or polymer coating layer includes an opening on the region of the TMD layer suspended over the nanogap, the opening leaving an exposed portion of the TMD layer therein, the opening dimensioned to accommodate only one enzyme molecule.

In various embodiments, the dielectric, ceramic or polymer layer comprises PMMA or SiO₂.

In various embodiments, the enzyme molecule comprises a DNA polymerase or an RNA polymerase.

In various embodiments, the enzyme molecule is attached to the TMD layer via a biotin-streptavidin linkage.

In various embodiments, the TMD layer includes as least one of vacancy defects, interstitial defects, and aggregated defects.

In various embodiments of the present disclosure, a DNA or RNA sequencing device comprises: an electrode array of conducting electrode pairs disposed on a substrate, each pair of electrodes comprising a source electrode and a drain electrode spaced-apart from the source electrode by a nanogap; a contiguous transition metal dichalcogenide (TMD) layer disposed on the first electrode and on the second electrode, the TMD layer configured as a bridge suspended over the nanogap; a polymerase enzyme molecule attached to a region of the TMD layer over the nanogap; and a microfluidic system encasing the conducting electrode pair, the contiguous TMD layer and the enzyme molecule attached thereto.

In various embodiments, the TMD layer comprises at least one TMD of structure MX₍₂₋ₓ₎ or MX₍₂₊ₓ₎, wherein X is S, Se or Te; x is 0-0.3; and M is Mo, W, Ti, Zr, Hf, V, Nb, Ta, Tc, Re, Co, Rh, Ir, Ni, Pd, or Pt.

In various embodiments, a single-molecule biosensor comprises: a conducting electrode pair disposed on a substrate, the electrode pair comprising a first electrode and a second electrode spaced-apart from the first electrode by a nanogap; a contiguous Si-material layer disposed on the first electrode and on the second electrode, the Si-material layer configured as a bridge suspended over the nanogap; an enzyme molecule attached to a region of the Si-material layer suspended over the nanogap; and a microfluidic system encasing the conducting electrode pair, the contiguous Si-material layer and the enzyme molecule attached thereto.

In various embodiments, the Si-material layer comprises a crystalline silicon, a p-type doped silicon semiconductor material or an n-type doped silicon semiconductor material.

In various embodiments, the substrate comprises SiO₂ or Al₂O₃.

In various embodiments, the pair of conducting electrodes comprise at least one of Au, Pt, Ag, Pd, Rh, Ru, or alloys thereof.

In various embodiments, the nanogap is from about 1 nm to about 50 nm.

In various embodiments, the single-molecule biosensor further comprises a dielectric, ceramic or polymer coating layer disposed on the Si-material layer on a side opposite the first and second electrodes, wherein the dielectric, ceramic or polymer coating layer includes an opening on the region of the Si-material layer suspended over the nanogap, the opening leaving an exposed portion of the Si-material layer therein, the opening dimensioned to accommodate only one enzyme molecule.

In various embodiments, the dielectric, ceramic or polymer layer comprises PMMA or SiO₂.

In various embodiments, the enzyme molecule comprises a DNA polymerase or an RNA polymerase.

In various embodiments, the enzyme molecule is attached to the Si-material layer via a biotin-streptavidin linkage.

In various embodiments, the Si-material layer was obtained from a silicon-on-insulator (SOI) wafer.

In various embodiments, the substrate and Si-Material layer comprise a silicon-on-insulator (SOI) wafer.

In various embodiments of the present disclosure, a DNA or RNA sequencing device comprises: an electrode array of conducting electrode pairs disposed on a substrate, each pair of electrodes comprising a source electrode and a drain electrode spaced-apart from the source electrode by a nanogap; a contiguous Si-material layer disposed on the first electrode and on the second electrode, the Si-material layer configured as a bridge suspended over the nanogap; a polymerase enzyme molecule attached to a region of the Si-material layer over the nanogap; and a microfluidic system encasing the conducting electrode pair, the contiguous Si-material layer and the enzyme molecule attached thereto.

In the detailed description, references to “various embodiments”, “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected, coupled or the like may include permanent (e.g., integral), removable, temporary, partial, full, and/or any other possible attachment option. Any of the components may be coupled to each other via friction, snap, sleeves, brackets, clips or other means now known in the art or hereinafter developed. Additionally, any reference to without contact (or similar phrases) may also include reduced contact or minimal contact.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

All structural, chemical, and functional equivalents to the elements of the above-described various embodiments that are known to those of ordinary skill in the art are expressly incorporated herein by reference and are intended to be encompassed by the present claims. Moreover, it is not necessary for an apparatus or component of an apparatus, or method in using an apparatus to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises”, “comprising”, or any other variation thereof, are intended to cover a non-exclusive inclusion, such that a chemical, chemical composition, process, method, article, or apparatus that comprises a list of elements does not include only those elements but may include other elements not expressly listed or inherent to such chemical, chemical composition, process, method, article, or apparatus.

1. A single-molecule biosensor comprising:
a conducting electrode pair disposed on a substrate, the electrode pair comprising a first electrode and a second electrode spaced-apart from the first electrode by a nanogap of fixed width;
a TMD, Si, or doped Si-semiconductor layer disposed on the substrate electrically connecting the first and second electrodes;
a dielectric layer disposed completely over the first and second electrodes to encase the first and second electrodes, and disposed partially over the TMD, Si, or doped Si-semiconductor layer so as to leave an exposed portion of the TMD, Si, or doped Si-semiconductor layer having a width less than the width of the nanogap;
an enzyme molecule attached to the exposed portion of the TMD, Si, or doped Si-semiconductor layer; and
a microfluidic system encasing the conducting electrode pair, the contiguous TMD layer and the enzyme molecule attached thereto.

2. The single-molecule biosensor of claim 1, wherein the TMD layer comprises at least one TMD having a structure MS₍₂₋ₓ₎, MS₍₂₊ₓ₎, MSe₍₂₋ₓ₎, MSe₍₂₊ₓ₎, MTe₍₂₋ₓ₎ or MTe₍₂₊ₓ₎, wherein x is 0-0.3, and M is Mo, W, Ti, Zr, Hf, V, Nb, Ta, Tc, Re, Co, Rh, Ir, Ni, Pd, or Pt.

3. The single-molecule biosensor of claim 1, wherein the TMD layer comprises a TMD selected from the group consisting of MoS₂, WS₂, TiS₂, ZrS₂, HfS₂, VS₂, NbS₂, TaS₂, TcS₂, ReS₂, CoS₂, RhS₂, IrS₂, NiS₂, PdS₂, PtS₂, MoSe₂, WSe₂, TiSe₂, ZrSe₂, HfSe₂, VSe₂, NbSe₂, TaSe₂, TcSe₂, ReSe₂, CoSe₂, RhSe₂, IrSe₂, NiSe₂, PdSe₂, PtSe₂, MoTe₂, WTe₂, TiTe₂, ZrTe₂, HfTe₂, VTe₂, NbTe₂, TaTe₂, TcTe₂, ReTe₂, CoTe₂, RhTe₂, IrTe₂, NiTe₂, PdTe₂, PtTe₂, and mixtures thereof.

4. The single-molecule biosensor of claim 1, wherein the TMD layer comprises a mixed TMD compound selected from the group consisting of Mo(SₓSe_{y}Te_{z})₂, W(SₓSe_{y}Te_{z})₂, Ti(SₓSe_{y}Te_{z})₂, Zr(SₓSe_{y}Te_{z})₂, Hf(SₓSe_{y}Te_{z})₂, V(SₓSe_{y}Te_{z})₂, Nb(SₓSe_{y}Te_{z})₂, Ta(SₓSe_{y}Te_{z})₂, Tc(SₓSe_{y}Te_{z})₂, Re(SₓSe_{y}Te_{z})₂, Co(SₓSe_{y}Te_{z})₂, Rh(SₓSe_{y}Te_{z})₂, Ir(SₓSe_{y}Te_{z})₂, Ni(SₓSe_{y}Te_{z})₂, Pd(SₓSe_{y}Te_{z})₂, Pt(SₓSe_{y}Te_{z})₂, and mixtures thereof, wherein (x+y+z) is 0.7-1.3.

5. The single-molecule biosensor of claim 1, wherein the TMD layer comprises at least one TMD compound of structure M_{(1-w)}N_{y}X_{(2-z)}Y_{z}, wherein M is Al, Si, Ga, Ge, In, Sn, Sb, Bi, Na, K, Ca, Mg, Sr, or Ba; X is S, Se, or Te; Y is Li, B, C, N, O, P, F, Cl, or I; w is 0-0.3; and z is 0-0.3.

6. The single-molecule biosensor of claim 1, wherein the substrate comprises Si, SiO₂ on Si, or Al₂O₃ on Si.

7. The single-molecule biosensor of claim 1, wherein the pair of conducting electrodes comprise at least one of Au, Ag, Pd, Pt, Ru, Rh, Al, Cu, Ni, or alloys thereof.

8. The single-molecule biosensor of claim 1, wherein the Si, or doped Si-semiconductor layer comprises single crystalline silicon, n-type doped silicon, or p-type doped silicon.

9. The single-molecule biosensor of claim 1, wherein the width of the nanogap is from about 20 nm to about 100 nm, and wherein the width of the exposed portion of the TMD, Si, or doped Si-semiconductor layer is about 5 nm.

10. The single-molecule biosensor of claim 1, wherein the enzyme molecule comprises a DNA polymerase or an RNA polymerase.

11. A DNA or RNA sequencing device comprising:
a pair of electrodes disposed on a substrate, the pair comprising a first electrode and a second electrode spaced-apart from the first electrode by a nanogap having a width W1;
a first nanopillar electrically attached to the first electrode and a second nanopillar electrically attached to the second electrode, wherein the two nanopillars are separated by a width W2 and wherein W2≥W1;
a dielectric layer disposed over the electrode pair and over the nanogap to surround the nanopillars such that only a top surface of each nanopillar is exposed;
a TMD, Si, or doped Si-semiconductor layer disposed on the dielectric layer and over the exposed top surface of each nanopillar, electrically connecting the first and second nanopillars;
an enzyme molecule attached to a region of the TMD, Si, or doped Si-semiconductor layer between the nanopillars and directly over the nanogap; and
a microfluidic system encasing the electrode pair, the TMD, Si, or doped Si-semiconductor layer and the enzyme molecule attached thereto.

12. The DNA or RNA sequencing device of claim 11, wherein the TMD layer comprises at least one TMD having a structure MS₍₂₋ₓ₎, MS₍₂₊ₓ₎, MSe₍₂₋ₓ₎, MSe₍₂₊ₓ₎, MTe₍₂₋ₓ₎ or MTe₍₂₊ₓ₎, wherein x is 0-0.3, and M is Mo, W, Ti, Zr, Hf, V, Nb, Ta, Tc, Re, Co, Rh, Ir, Ni, Pd, or Pt.

13. The DNA or RNA sequencing device of claim 11, wherein the TMD layer comprises a TMD selected from the group consisting of MoS₂, WS₂, TiS₂, ZrS₂, HfS₂, VS₂, NbS₂, TaS₂, TcS₂, ReS₂, CoS₂, RhS₂, IrS₂, NiS₂, PdS₂, PtS₂, MoSe₂, WSe₂, TiSe₂, ZrSe₂, HfSe₂, VSe₂, NbSe₂, TaSe₂, TcSe₂, ReSe₂, CoSe₂, RhSe₂, IrSe₂, NiSe₂, PdSe₂, PtSe₂, MoTe₂, WTe₂, TiTe₂, ZrTe₂, HfTe₂, VTe₂, NbTe₂, TaTe₂, TcTe₂, ReTe₂, CoTe₂, RhTe₂, IrTe₂, NiTe₂, PdTe₂, PtTe₂, and mixtures thereof.

14. The DNA or RNA sequencing device of claim 11, wherein the TMD layer comprises a mixed TMD compound selected from the group consisting of Mo(SₓSe_{y}Te_{z})₂, W(SₓSe_{y}Te_{z})₂, Ti(SₓSe_{y}Te_{z})₂, Zr(SₓSe_{y}Te_{z})₂, Hf(SₓSe_{y}Te_{z})₂, V(SₓSe_{y}Te_{z})₂, Nb(SₓSe_{y}Te_{z})₂, Ta(SₓSe_{y}Te_{z})₂, Tc(SₓSe_{y}Te_{z})₂, Re(SₓSe_{y}Te_{z})₂, Co(SₓSe_{y}Te_{z})₂, Rh(SₓSe_{y}Te_{z})₂, Ir(SₓSe_{y}Te_{z})₂, Ni(SₓSe_{y}Te_{z})₂, Pd(SₓSe_{y}Te_{z})₂, Pt(SₓSe_{y}Te_{z})₂, and mixtures thereof, wherein (x+y+z) is 0.7-1.3.

15. The DNA or RNA sequencing device of claim 11, wherein the TMD layer comprises at least one TMD compound of structure M_{(1-w)}N_{y}X_{(2-z)}Y_{z}, wherein M is Al, Si, Ga, Ge, In, Sn, Sb, Bi, Na, K, Ca, Mg, Sr, or Ba; X is S, Se, or Te; Y is Li, B, C, N, O, P, F, Cl, or I; w is 0-0.3; and z is 0-0.3.

16. The DNA or RNA sequencing device of claim 11, wherein the dielectric layer comprises PMMA, SiO₂ or HSQ.

17. The DNA or RNA sequencing device of claim 11, wherein the first and second electrodes comprise at least one of Au, Ag, Pd, Pt, Ru, Rh, Al, Cu, Ni, or alloys thereof, and wherein the first and second nanopillars comprise Au, Pd, Pt, Ru, or Rh.

18. The DNA or RNA sequencing device of claim 11, wherein the Si, or doped Si-semiconductor layer comprises single crystalline silicon, n-type doped silicon, or p-type doped silicon.

19. The DNA or RNA sequencing device of claim 11, wherein W1 is from about 5 nm to about 20 nm, and wherein W2 is from about 5 nm to about 100 nm.

20. The DNA or RNA sequencing device of claim 11, wherein the enzyme molecule comprises a DNA polymerase or an RNA polymerase.
